(19) United States
(12) Patent Application Publication
(10) Pub. No.: US 2003/0100467 A1
(43) Pub. Date: May 29, 2003

(54) Binding phenol oxidizing enzyme-peptide complexes

(76) Inventors: Wolfgang Aehle, Delfgauw (NL); Toby M. Baldwin, Palo Alto, CA (US); Franciscus J. C. van Gastel, Union City, CA (US); Giselle G. Janssen, San Carlos, CA (US); Christopher J. Murray, Soquel, CA (US); Huaming Wang, Fremont, CA (US); Deborah S. Winetzky, Foster City, CA (US)

Correspondence Address: GENENCOR INTERNATIONAL, INC., 925 PAGE MILL ROAD, PALO ALTO, CA, 94304, US

(21) Appl. No.: 09/954,385

(22) Filed: Sep. 12, 2001

(51) Int. Cl. C11D 3/00; C11D 3/386; C12N 9/00

(52) U.S. Cl. 510/392; 510/530; 435/183

(57) ABSTRACT

The present application relates to peptides which bind to a target stain, phenol oxidizing enzyme-binding peptide complexes wherein the binding peptide is attached to the C-terminus of the phenol oxidizing enzyme or is inserted or substituted into the phenol oxidizing enzyme. In a preferred embodiment the phenol oxidizing enzyme is a laccase specifically Stachybotrys oxidase B and variants thereof. The invention provides expression vectors comprising the phenol oxidizing enzyme-binding peptide complex as well as host cells comprising the vectors.

### FIELD OF THE INVENTION

The present invention relates to peptides which bind to a selective target stain and to a phenol oxidizing enzyme-peptide complex, which includes the binding peptide conjugated with a phenol-oxidizing enzyme. The phenol oxidizing enzyme-peptide complex may be used in enzymatic compositions, particularly detergent compositions to specifically target stains.

### BACKGROUND OF THE INVENTION

Phenol oxidizing enzymes function by catalyzing redox reactions, i.e., the transfer of electrons from an electron donor (usually a phenolic compound) to molecular oxygen (which acts as an electron acceptor) which is reduced to H₂O or H₂O₂. While being capable of using a wide variety of different phenolic compounds as electron donors, phenol oxidizing enzymes are very specific for molecular oxygen as the electron acceptor.

Phenol oxidizing enzymes can be utilized for a wide variety of applications, including in the detergent industry, the paper and pulp industry, the textile industry, and the food industry. Phenol oxidizing enzymes are specifically used for their color modifying ability for example for pulp and paper bleaching, for bleaching the color of stains on fabric, and for anti-dye transfer in detergent and textile applications. While the prior art does teach various phenol oxidizing enzymes useful in the above mentioned applications, there remains a need for new phenol oxidizing enzymes that have stain bleaching ability and anti-dye transfer properties. It is a purpose of the present application to create phenol oxidizing enzyme complexes with increased binding ability to target stains. A further purpose of the present invention is to provide a phenol oxidizing enzyme complex having bleaching ability.

### SUMMARY OF THE INVENTION

In one aspect the invention pertains to a binding peptide having an amino acid sequence illustrated in any one of SEQ ID NOS: 2 through 433 wherein the binding peptide selectively binds to a colored substance. In one preferred embodiment the binding peptides are the peptides listed in Table 1. In another preferred embodiment the binding peptides further include a cysteine amino acid residue added to each end of the binding peptide. In a third preferred embodiment the binding peptides bind to a carotenoid stain.

In a second aspect, the invention pertains to a binding peptide comprising a repeatable motif of 3 to 6 amino acids. In one preferred embodiment, the repeatable motif is selected from the group consisting of SAPA, TAPP, APAL, PPP, PPPP, SSPH, SSP, SSK, SPT, LPAQ, PPPL, PTPL, SPTT, PLVP, PLP, YTKP, SLH, SLLNA, SPL, SNLA, SPLTQ, TTT, MRND, AARN, ARND, LSPG, NPNN, NLAT, NTS, PHSM, PPWM, PTSP, TGGA, YLPS, YTKP, PGSL, APS, TPV, TTTS and LNAT, wherein the binding peptide has 6 to 15 amino acid residues and binds to a carotenoid chromophore stain on a fabric.

In a third aspect, the invention pertains to polynucleotides encoding the binding peptides.

In a fourth aspect, the invention pertains to a phenol oxidizing enzyme-peptide complex comprising a phenol oxidizing enzyme and a peptide having an amino acid sequence illustrated in any one of SEQ ID NOS: 2 through 433 or a peptide having a repeatable motif as illustrated in Table 2, wherein the complex binds to a colored substance. In one preferred embodiment the phenol oxidizing enzyme is a laccase and most preferably the laccase is obtainable from a Stachybotrys species. In a further preferred embodiment the laccase has the amino acid sequence illustrated in SEQ ID NO: 1. In another preferred embodiment the binding peptide is attached to the C-terminus of the phenol oxidizing enzyme. In yet another preferred embodiment the binding peptide is combined with the phenol oxidizing enzyme in an internal site, preferably by insertion or substitution.

In a fifth aspect, the invention pertains to expression vectors and host cells incorporating the expression vectors comprising a polynucleotide encoding a phenol oxidizing enzyme-peptide complex or a polynucleotide encoding the binding peptides according to the invention. In one preferred embodiment the host cell is a fungal cell.

In a sixth aspect, the invention pertains to a method of enhancing the binding of a laccase enzyme to a target stain. The method includes obtaining a binding peptide of the invention, combining the peptide with a laccase to form a laccase-peptide complex, and exposing a target stain to the laccase-peptide complex under suitable conditions to allow the complex to bind with the target stain.

In a seventh aspect, the invention pertains to detergent and enzyme compositions comprising one or more surfactants and/or additives and the phenol oxidizing enzyme-peptide complex of the invention, wherein said complex selectively binds to a target stain during a wash cycle that includes agitation. In one preferred embodiment the phenol oxidizing enzyme is a laccase. In another preferred embodiment the compositions include one or more enzymes other than laccase.

In an eighth aspect, the invention pertains to a method for producing a host cell comprising a polynucleotide encoding a laccase-peptide complex, comprising (a) obtaining a polynucleotide encoding a laccase having at least 68% identity to the amino acid sequence disclosed in SEQ ID NO: 1; (b) obtaining a polynucleotide encoding a binding peptide having an amino acid sequence as illustrated in any one SEQ ID NOS: 2-433; conjugating the polynucleotide of (a) with (b); introducing said conjugated polynucleotide into a host cell; and growing said host cell under conditions suitable for the production of said laccase-peptide complex.

In a ninth aspect, the invention pertains to a method of using a binding peptide to target a stain on a textile comprising obtaining a binding peptide as illustrated in any one of SEQ ID NOS: 2-433; and exposing said binding peptide to a target stain, wherein said binding peptide binds to said stain and not to said textile.

In a tenth aspect, the invention pertains to a method of enhancing the selectivity of a phenol oxidizing enzyme to a target stain which comprises, derivatizing a laccase with a binding peptide as illustrated in any one of SEQ ID NOS: 2-433 to form a laccase-peptide complex; and exposing the laccase-peptide complex to a target stain, wherein selectivity of the laccase-peptide complex to the target stain is greater than the selectivity of the a nonderivatized laccase having the same amino acid sequence as the laccase of the laccase-peptide complex.

### BRIEF DESCRIPTION OF THE DRAWINGS

FIG. 1 is a list of binding peptides SEQ ID NOs: 2-433 according to the invention that selectively bind to tomato or paprika stains on cotton using either a cyclic 7-mer (FIGS. 1A and 1C), a linear 12-mer (FIGS. 1B and 1D) or mixed population (FIG. 1E) of a phage random peptide library as further discussed in the examples.

FIG. 2 illustrates the amino acid sequence (SEQ ID NO: 1) for the enzyme designated herein as the Stachybotrys phenol oxidase B having MUCL accession number 38898. (Also reference is made to U.S. Pat. No. 6,168,936)

FIG. 3 provides an illustration of the vector pGAPT which was used for the expression of Stachybotrys phenol oxidase B (SEQ ID NO: 1) and derivatives thereof in either derivatized form (as a laccase-peptide complex) or in nonderivatized form (the laccase backbone with no binding peptide combination) in Aspergillus niger. Base 1 to 1134 contains Aspergillus niger glucoamylase gene promoter. Base 1227 to 1485 and 3079 to 3100 contains Aspergillus niger glucoamylase terminator. Aspergillus nidulans pyrG gene was inserted from 1486 to 3078 as a marker for fungal transformation. The rest of the plasmid contains pUC18 sequence for propagation in E. coli. Nucleic acid encoding the Stachybotrys phenol oxidase B of SEQ ID NO: 1 was cloned into the BGI II and Xba I restriction sites.

FIG. 4 illustrates the scheme for C-terminus insertion of a binding peptide in Stachybotrys phenol oxidase B.

FIG. 5 illustrates the preferential binding of peptide YGYLPSR (SEQ ID NO: 16) to tomato stained cotton swatches.

FIG. 6 illustrates the oxidation of ABTS by laccase-peptide complexes: (a) SEQ ID NO: 1 and IERSAPATAPPP (SEQ ID NO: 92); (b) SEQ ID NO: 1 and KASAPAL (SEQ ID NO: 24); (c) SEQ ID NO: 1 and the C-C derivative of SEQ ID NO: 24; and (d) SEQ ID NO: 1.

FIG. 7 compares the binding of a variant of laccase (SEQ ID NO: 1) wherein the binding peptide YGYLPSR (SEQ ID NO: 16) is attached to the C-terminus and the laccase includes the amino acid substitution set M254F/E346V/E348Q to the non-derivatized laccase M254F/E346V/E348Q on tomato and non-stained cotton.

### DETAILED DESCRIPTION OF THE INVENTION

General Terms

Unless defined otherwise, all technical and scientific terms used herein have the same meaning as commonly understood by one of ordinary skill in the art to which this invention pertains. For the purpose of the present invention, the following terms are used to describe the invention herein.

The term “peptide” refers to an oligomer in which the monomer units are amino acids (typically, but not limited to L-amino acids) linked by an amide bond. Peptides may be two or more amino acids in length. Peptides that are greater than 100 amino acids in length are generally referred to as polypeptides. However, the terms, peptide, polypeptide and protein may be used interchangeably. Standard abbreviations for amino acids are used herein and reference is made to Singleton et al., (1987) Dictionary of Microbiology and Molecular Biology, 2nd Ed. page 35.

“Percent sequence identity” with respect to peptide or polynucleotide sequences refers to the percentage of residues that are identical in the two sequences. Thus 95% amino acid sequence identity means that 95% of the amino acids in the sequences are identical. Percent identity can be determined by direct comparison of the sequence information provided between two sequences and can be determined by various commercially available computer programs such as BESTFIT, FASTA, TFASTA and BLAST.

A “binding peptide” according to the invention is a peptide that binds to a target with a binding affinity of at least about 10⁻² M, at least about 10⁻³ M, at least about 10⁻⁴ M, at least about10⁻⁵ M and preferably between about 10⁻² M to 10⁻¹⁵ M.

The binding affinity of a peptide for its target or a phenol oxidizing enzyme-peptide complex for its target may be described by the dissociation constant (K_{D}). K_{D} is defined by k_{off}/kₒₙ. The k_{off} value defines the rate at which a bound-target complex breaks apart or separates. This term is sometimes referred to in the art as the kinetic stability of the peptide-target complex or the ratio of any other measurable quantity that reflects the ratio of binding affinity such as an enzyme-linked immunosorbent assay (ELISA) signal. Kn describes the rate at which the target and the peptide (or the enzyme-peptide complex) combine to form a bound-target complex. In one aspect, the k_{off} value for the bound-target complex will be less that about 10⁻² sec⁻¹, less that about 10⁻³ sec⁻¹, less than about 10⁻⁴ sec⁻¹ and also less than about 10⁻⁵ sec⁻¹.

Selectivity is defined herein as enhanced binding of a peptide or protein to a target compared to the binding of the peptide or protein to a non-target. Selectivity may also be defined as the enhanced binding of a derivatized phenol oxidizing enzyme to a target compared to the binding of a nonderivatized phenol oxidizing enzyme to a target. Selectivity may be in the range of about 1.25:1 to 25:1; about 1.5:1 to 15:1; about 1.5:1 to 10:1; and about 1.5:1 to 5:1. Preferably the selectively is at least 2:1 for either a) the binding of the peptide to a target compared to the binding to a non-target or b) the binding of a derivatized phenol oxidizing enzyme to a target compared to the binding of the nonderivatized phenol oxidizing enzyme to a target.

As used herein a phenol oxidizing enzyme refers to those enzymes which are capable of catalyzing redox reactions wherein the electron donor is usually a phenolic compound and which are specific for molecular oxygen or hydrogen peroxide as the electron acceptor. Examples of such enzymes are laccases (EC1.10.3.2), bilirubin oxidases (EC1.3.3.5), phenol oxidases (EC 1.14.18.1) and catechol oxidases (EC 1.10.3.1). Preferred phenol oxidizing enzymes are laccases. The phenol oxidizing enzymes useful according to the invention may be naturally occurring or recombinant enzymes.

A recombinant phenol-oxidizing enzyme is one in which a nucleic acid sequence encoding the enzyme is modified to produce a variant nucleic acid sequence which encodes the substitution, deletion or insertion of one or more amino acids in the naturally occurring amino acid sequence. Phenol oxidizing enzyme variants may include the mature form of the enzyme variant, as well as the pro- and prepro-forms of such variants and post-translational modification such as glycosylation.

A “phenol oxidizing enzyme-peptide complex” means a phenol-oxidizing enzyme combined with a binding peptide according to the invention, and is also referred to as a derivatized enzyme. A “laccase-peptide complex” means a laccase enzyme combined with a binding peptide according to the invention. The binding peptide may be combined with the phenol oxidizing enzyme by various means, for example; the binding peptide may be attached to the C-terminus or the N-terminus of the enzyme. The binding peptide may replace an internal sequence of the enzyme or be inserted into an internal sequence of the enzyme or any combination thereof. Additionally, more than one copy of the same or different binding peptides may be combined with the phenol oxidizing enzyme of interest. A non-derivatized phenol oxidizing enzyme is one wherein a binding peptide has not been combined with the phenol oxidizing enzyme.

A stain is defined herein as a colored compound which is the target for oxidation by phenol-oxidizing enzymes. A coloured compound is a substance that adds colour to a textile or to substances which result in the visual appearances of stains. Targeted classes of coloured substances which may appear as a stain may include the following; a) porphyrin derived structures, such as heme in blood stain or chlorophyll in plants; b) tannins and polyphenols (see P. Ribéreau-Gayon, Plant Phenolics, Ed. Oliver & Boyd, Edinburgh, 1972, pp.169-198) which occur in tea stains, wine stains, banana stains, and peach stains; c) carotenoids and carotenoid derivatives, the coloured substances which occur in tomato (lycopene, red), mango (carotene, orange-yellow) and paprika. Also included are the oxygenated carotenoids, xanthophylls (G. E. Bartley et al., The Plant Cell (1995), Vol 7, 1027-1038); d) anthocyanins, the highly coloured molecules which occur in many fruits and flowers (P. Ribéreau-Gayon, Plant Phenolics, Ed. Oliver & Boyd, Edinburgh, 1972, 135-169); and e) Maillard reaction products, the yellow/brown coloured substances which appear upon heating of mixtures of carbohydrate molecules in the presence of protein/peptide structures, such as found in cooking oil. A coloured compound may also be a dye that is incorporated into a fiber by chemical reaction, adsorption or dispersion. Examples include direct Blue dyes, acid Blue dyes, reactive Blue dyes, and reactive Black dyes. Particularly preferred targets of the invention include carotenoid and xanthophyll stains.

The phrase “modify the colour associated with a coloured compound” means that the coloured compound is changed through oxidation, either directly or indirectly, such that the colour appears modified i.e. the colour visually appears to be increased, decreased, decoloured, bleached or removed, particularly bleached.

As used herein the term “enhancer” or “mediator” refers to any compound that is able to modify the colour associated with a coloured compound in association with a phenol-oxidizing enzyme or a compound which increases the oxidative activity of the phenol oxidizing enzyme. The enhancing agent is typically an organic compound.

As used herein, Stachybotrys refers to any Stachybotrys species which produces a phenol oxidizing enzyme and particularly a laccase enzyme capable of modifying the colour associated with coloured compounds. The present invention encompasses derivatives of natural isolates of Stachybotrys including progeny, mutants or variants as long as the derivative is able to produce a phenol oxidizing enzyme, and particularly a laccase, capable of modifying the colour associated with coloured compounds.

As used in the specification and claims, the singular “a”, “an” and “the” include the plural references unless the context clearly dictates otherwise. For example, the term a vector may include a plurality of vectors.

The following references describe the general techniques employed herein: Sambrook et al (1989) Molecular Cloning: A Laboratory Manual, Cold Spring Harbour Laboratory Press, Cold Spring Harbour, N.Y.; and Ausubel et al. (1987) Current Protocols in Molecular Biology, Greene-Publishing & Wiley Interscience NY (Supplemented through 1999).

The contents of all references, patents and published patent applications cited throughout this application are hereby incorporated by reference in their entirety.

B. Binding Peptides

The binding peptides of the invention may be obtained using methods well known in the art. Preferably the binding peptides are identified by using random peptide libraries which are screened using techniques including phage display, biopanning and acid elution. These techniques are described in various references such as, Scott and Smith (1990) Science 249:386; Smith and Scott (1993) Methods Enzymol. 217:228; Cwirla et al., (1990) Proc. Natl. Acad. Sci. USA 87:6378; Parmley et al., (1988) Gene 73:305; Balass et al., (1996) Anal. Biochem., 243:264 and Huls et al., (1996) Nature Biotechnol., 7:276).

While a random peptide library is a preferred library used to identify binding peptides according to the invention, the binding peptides useful in the invention are not limited to identification using a random peptide library. Binding peptides of the invention may be identified from use of synthetic peptide libraries, peptide loop libraries, antibody libraries and protein libraries. Those skilled in the art are aware of commercially available libraries from sources such as New England BioLabs and Dyax Corporation.

While phage display is the preferred method used to screen peptides other display methods may also be used for example, yeast display and ribosome display.

Once the peptide library is screened, the peptides that bind to a specific target may be identified by various means that are well known including, acid elution, polymerase chain reaction (PCR), sequencing, and other well-known methods.

Preferably the binding peptides of the invention are between 4 and 50 amino acids in length, also between 4-25 amino acids in length, between 4-20 amino acids in length and between 6-15 amino acids in length.

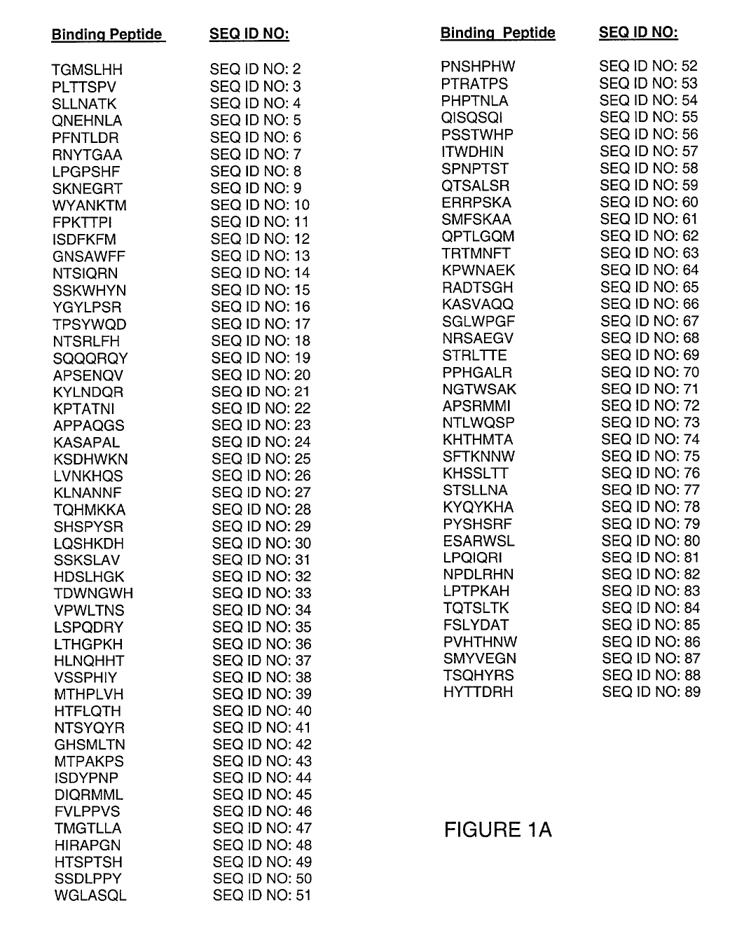

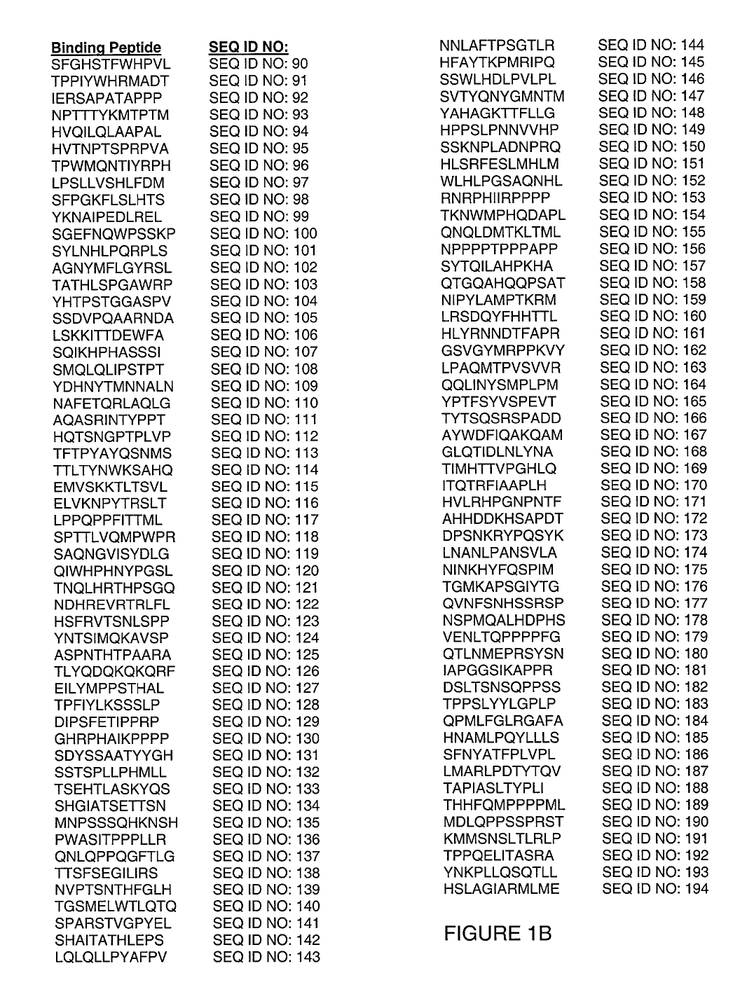

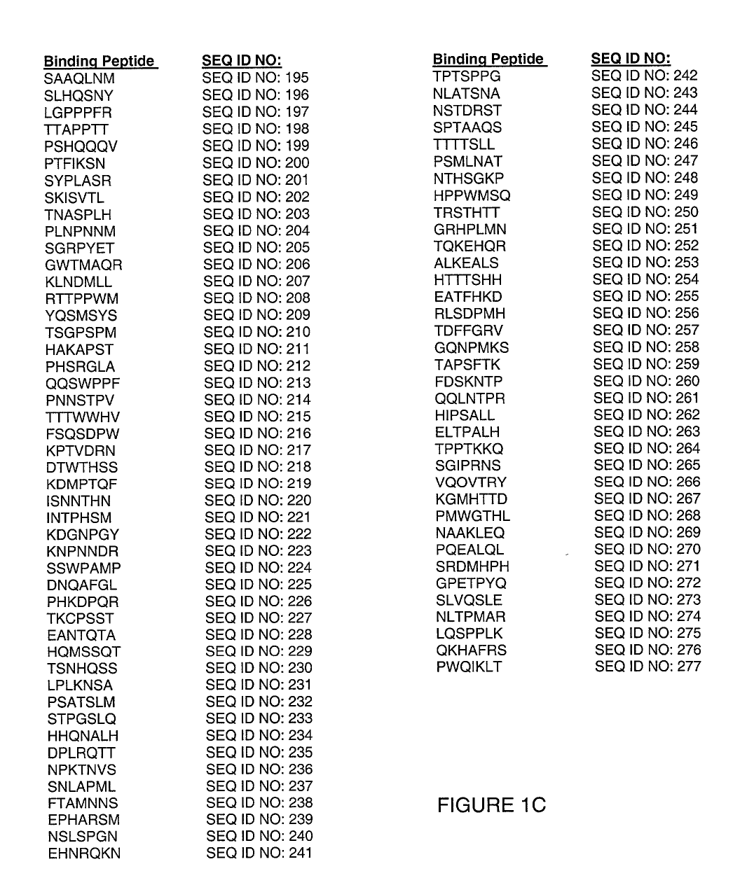

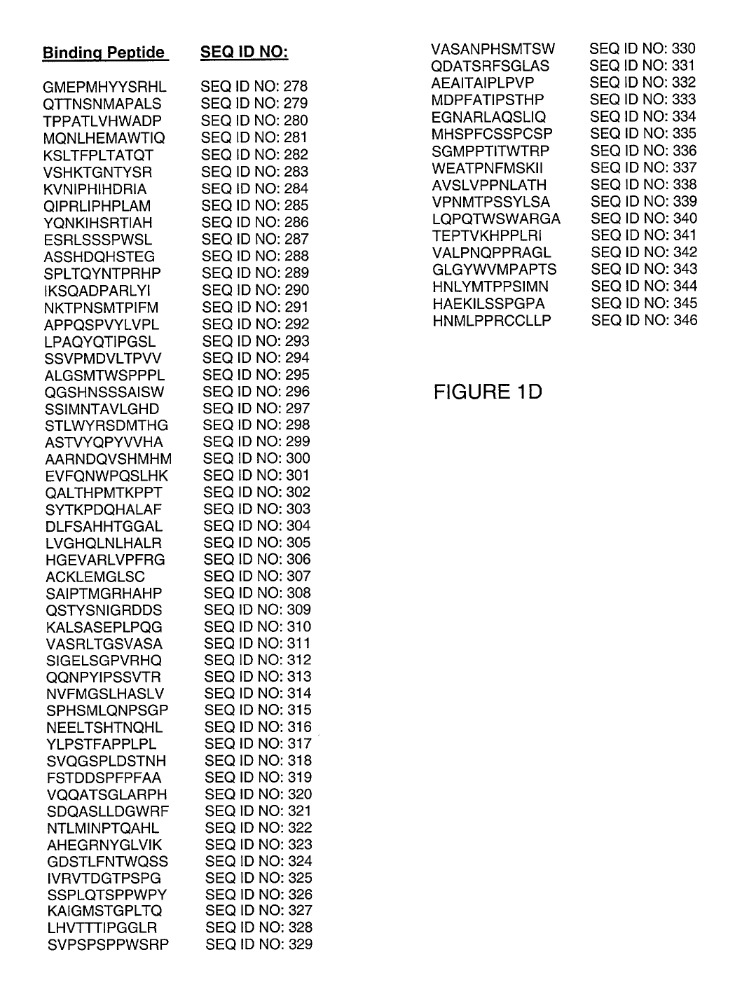

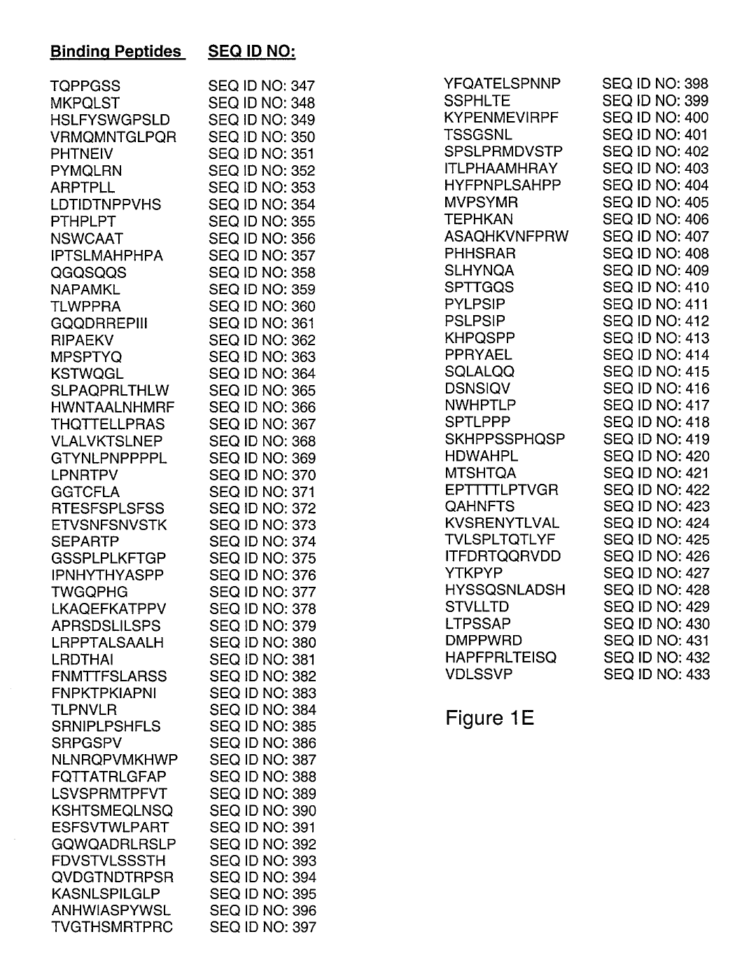

The binding peptides according to the invention include the peptides listed in FIGS. 1A-E (SEQ ID NOS: 2-433). In one embodiment, preferred binding peptides are listed in Table 1.

TABLE 1

| | |
|---|---|
| SLLNATK | SEQ ID NO:4 |
| YGYLPSR | SEQ ID NO:16 |
| KASAPAL | SEQ ID NO:24 |
| IERSAPATAPPP | SEQ ID NO:92 |
| HVQILQLAAPAL | SEQ ID NO:94 |
| YHTPSTGGASPV | SEQ ID NO:104 |
| SSDVPQAARNDA | SEQ ID NO:105 |
| QIWHPHNYPGSL | SEQ ID NO:120 |
| TTAPPTT | SEQ ID NO:198 |
| STPGSLQ | SEQ ID NO:233 |
| PSMLNAT | SEQ ID NO:247 |
| QTTNSNMAPALS | SEQ ID NO:279 |
| LPAQYQTIPGSL | SEQ ID NO:293 |
| AARNDQVSHMHM | SEQ ID NO:300 |
| DLFSAHHTGGAL | SEQ ID NO:304 |
| YLPSTFAPPLPL | SEQ ID NO:317 |

Particularly preferred binding peptides are SEQ ID NOS: 4, 16, 24, 92 and 317.

In a further embodiment, the peptides according to the invention may include cysteine residues on each end of the binding peptide and are referred to herein as binding peptide C-C derivatives. For example, the binding peptide PSMLNAT may also exist in the form CPSMLNATC and is considered a binding peptide according to the invention. When a binding peptide according to the invention is used as an internal replacement or insert for internal loops or turns in the phenol oxidizing enzyme, the binding peptide may be used in the C-C derivative form or non C-C derivative form. While any of the peptides listed in FIG. 1 may include the C-C derivatized form, particularly preferred are the peptides listed in FIGS. 1A and 1C. Additionally, the amino acid residue triad GGH or GGHGG may be added to either end of the binding peptides according to the invention.

The invention further includes binding peptides having at least 60% but less than 100% amino acid sequence identity to the binding peptides listed in FIG. 1. For example at least 65%, at least 70%, at least 75%, at least 80%, at least 85%, at least 90%, at least 93%, at least 95%, at least 97%, at least 99% amino acid sequence identity. A peptide having at least 60% sequence identity to a binding peptide listed in FIG. 1 will also have a binding affinity for its target in the range of 10⁻²M to 10⁻¹⁵M, generally at least about 10⁻²M, at least about 10⁻³M, at least about 10⁻⁴M and at least about 10⁻⁵M.

In another embodiment, binding peptides according to the invention may have a repeatable motif of at least three amino acid residues in common with the binding peptides listed in FIG. 1. However, the repeatable motif may include four, five or six amino acid residues. Repeatable motifs of the binding peptides include the following amino acid residues as listed in Table 2. Also included in Table 2 are sequence identifiers for representative binding peptides of FIG. 1 which include said repeatable motif.

TABLE 2

| CONSENSUS | Binding Peptide | CONSENSUS | Binding Peptide |
|---|---|---|---|
| SEQUENCE | SEQ ID NO: | SEQUENCE | SEQ ID NO: |
| AARND | 105, 300 | PPWM | 208, 249 |
| APAL | 24, 94, 279 | SAPA | 24, 92 |
| AARN | 105, 300 | LNAT | 4, 247 |
| ARND | 105, 300 | LSPG | 103, 240 |
| SPL | 132, 289, 326, | PPPP | 127, 153, 156, |
| | 372, 375, 425 | | 179, 186 |
| LTQ | 179, 289, 327, | PAR | 141, 290, 374, |
| | 425 | | 391 |
| NTSI | 14, 124 | TAPP | 92, 198 |
| PTSP | 95, 242 | TGGA | 104, 304 |
| PSST | 56, 227 | NPNN | 204, 223 |
| SLLNA | 4, 77 | PGN(C) | 48, 240 |
| SSP | 38, 190, 326, | PLP | 164, 310, 317, |
| | 375, 399, 419 | | 332, 385 |
| SPLTQ | 289, 425 | PLVP | 112, 186, 332 |
| TATHL | 103, 142 | PPPF | 179, 197 |
| NTS | 14, 18, 41, | PQSP | 292, 412 |
| | 124 | | |
| SPT | 49, 118, 245, | PSAT | 158, 232 |
| | 410 | | |
| LPAQ | 163, 293, 365 | PART | 374, 391 |
| PGSL | 120, 233, 293 | PPSSP | 190, 419 |
| PHSM | 221, 315, 330 | YTKP | 145, 303, 427 |
| PLTQ | 289, 327 | ALH(C) | 234, 263 |
| PPPL | 136, 295, 369 | ALSA | 310, 380 |
| YLPS | 16, 317 | (C)APS | 20, 72, 211, |
| | | | 259 |
| PSTH | 127, 333 | (C)ISD | 12, 44 |
| PTPL | 112, 353, 417 | (C)KAS | 24, 66 |
| PTTT | 93, 422 | (C)KLN | 27, 207 |
| QLQL | 108, 143 | (C)KPT | 22, 217 |
| RLAQ | 110, 334 | (C)LQS | 30, 193, 275 |
| (C)TTT | 93, 215, 246, | (C)SLH | 2, 32, 98, 196, |
| | 254, 328 | | 301, 314 |
| SIMN | 297, 344 | (C)SSK | 15, 31, 100, |
| | | | 150 |
| SNLA | 237, 428 | SAQN | 119, 152 |
| SPTT | 118, 410 | HSML | 42, 315 |
| SPV(C) | 3, 292 | IPST | 108, 333 |
| SSVP | 294, 433 | KAPS | 176, 211 |
| TFAP | 161, 317 | LNAN | 27, 174 |
| TFPL | 185, 281 | LPLK | 231, 375 |
| LPQR | 49, 100 | TIPG | 293, 328 |
| LSSS | 286, 392 | TPV(C) | 163, 214, 294 |
| LVPL | 185, 291 | TSHT | 316 |
| NLAT | 242, 339 | TSLL | 77, 246 |
| NPTS | 57, 94 | TSLM | 232, 357 |
| VASA | 310, 329 | TSPP | 242, 326 |
| NFSN | 176, 372 | ESFS | 372, 391 |
| AITA | 133, 141 | DVST | 393, 402 |
| PPSL | 148, 182 | IPLP | 332, 385 |
| NFSN | 176, 372 | PSLP | 149, 399 |
| NPKT | 235, 382 | SFTK | 75, 259 |
| PPRA | 341, 359 | SGLA | 320, 331 |
| SSPH | 37, 398, 418 | SSPL | 326, 375 |
| THPL | 38, 358 | TQPP | 179, 347 |
| TPSS | 338, 429 | SPPW | 326, 329 |
| PRLT | 364, 431 | SRSP | 166, 177 |
| KHPP | 340, 418 | MHTT | 169, 227 |
| STVL | 392, 428 | TTTT | 246, 422 |
| GLAS | 50, 330 | SNLSP | 123, 395 |

Particularly preferred repeatable motifs include SAPA, TAPP, APAL, PPP, PPPP, SSPH, SSP, SSK, SPT, LPAQ, PPPL, PTPL, SPTT, PLVP, PLP, YTKP, SLH, SLLNA, SPL, SNLA, SPLTQ, TTT, AARND, AARN, ARND, LSPG, NPNN, NLAT, NTS, PHSM, PPWM, PTSP, TGGA, YLPS, YTKP, PGSL, APS, TPV, TTTS and LNAT. More particularly preferred are SAPA, TAPP, APAL, PHSM, YLPS, AARND, ARND, SLLNA, PPPP, SNLA and NLAT. The repeatable motif may also include a cysteine residue at the beginning and/or end of the motif, for example SPV (SPVC); TPV (TPVC); SLH (CSLH); LQS (CLQS) and KAS (CKAS). Particularly preferred are (C)SLH, (C)TTT, (C)SSK, (C)LQS, and TPV(C).

In general, the repeatable motifs may occur alone, as multiple motifs in the same peptide, in sequential order, or overlapping one another. For example the binding peptide HVQILQLAAPAL (SEQ ID NO: 94) includes the repeatable motif APAL. The binding peptide YGYLPSR (SEQ ID NO: 16) includes the repeatable motif YLPS. The binding peptides SLLNATK (SEQ ID NO: 3) and PSMLNAT (SEQ ID NO: 247) include the repeatable motif LNAT. The binding peptide TTAPPTT (SEQ ID NO: 198) includes the repeatable motif TAPP. The binding peptides INTPHSM (SEQ ID NO: 221), SPHSMLQNPSGP (SEQ ID NO: 315) and VASANPHSMTSW (SEQ ID NO: 330) include the repeatable motif PHSM. The binding peptides VASANPHSMTSW (SEQ ID NO: 330), ESFSVTWLPART (SEQ ID NO: 391), and LPAQYQTIPGSL (SEQ ID NO: 297) include multiple motifs, two repeatable motifs, in the same sequence. The binding peptide IERSAPATAPPP (SEQ ID NO: 92) includes two repeatable motifs (SAPA and TAPP) in sequential order. The binding peptide KASAPAL (SEQ ID NO: 24) includes two overlapping repeatable motifs (SAPA and APAL).

Peptides sharing a repeatable motif with any one of the binding peptides of FIG. 1 will include 6-25 amino acid residues and preferably will include 6-15 amino acid residues. Further the peptides including a repeatable motif will bind to a target with a binding affinity similar to the binding affinity of the binding peptides of FIG. 1. Preferably the target will be a stain, preferably a carotenoid stain and the binding affinity will be at least about 10⁻²M, about 10⁻³M, about 10⁻⁴M, about 10⁻⁶M and generally between about 10⁻²M and 10⁻⁹M. These peptides are also considered binding peptides according to the invention and are referred to herein as homologous motif binding peptides. A homologous motif binding peptide will include not only a repeatable motif as defined herein, but will have between 20% and 95% sequence identity with a sequence illustrated in FIG. 1, that is at least 25% sequence identity, at least 30% sequence identity, at least 40% sequence, at least 50% sequence identity, at least 60% sequence identity to a binding peptide illustrated in FIG. 1 which includes the same repeatable motif. Preferably if the homologous motif binding peptide is a 7 amino acid residue peptide, the peptide will have at least 30% sequence identity with a binding peptide illustrated in FIG. 1 having the same repeatable motif when the peptides are aligned with no gaps. If the homologous motif binding peptide is a 12 amino acid residue peptide, the peptide will have at least 25% sequence identity with a binding peptide illustrated in FIG. 1 having the same repeatable motif when the peptides are aligned with no gaps.

In one embodiment, binding peptides having identical repeatable motifs may bind to stains with structurally and/or biochemically related chromophores with about the same binding affinity. Preferably in one aspect, the homologous motif binding peptides including one or more repeatable motifs will bind to the carotenoids, such as lycopene and beta-carotene. In another aspect, the peptides having one or more identical repeatable motifs will bind to the xanthophylls, such as casporubin and capsoxanthins.

Additionally binding peptides of the invention may include peptides having sequence clusters. A sequence cluster is defined herein as including a repeatable motif followed by 1 or 2 identical amino acid residues, wherein the repeatable motif and the identical amino acid residues are separated by 1 to 10, preferably 1 to 3 amino acids residues. Numerous examples of sequence clusters may be found in FIG. 1. Two such examples are SEQ ID NOS 103 and 142 wherein the repeatable motif TATHL is separated from the amino acid residue P by one amino acid residue and SEQ ID NOS: 93 and 422 wherein the repeatable motif PTTT is separated from the amino acid residue T by three amino acid residues.

The binding peptides according to the invention may be made by various well known techniques in the art and include chemical synthesis, PCR, and amplification.

C. Polynucleotides Encoding the Binding Peptides

The present invention encompasses polynucleotides which encode binding peptides according to the invention. Specifically polynucleotides include nucleic acid sequences encoding peptides illustrated in FIG. 1 (SEQ ID NOs: 2-433) and their C-C derivatives. Particularly preferred polynucleotides encode the binding peptides illustrated in Table 1 and their C-C derivatives. Additionally, polynucleotides which encode homologous motif binding peptides having identical repeatable motifs as those listed in Table 2 are part of the invention. As will be understood by the skilled artisan, due to the degeneracy of the genetic code, a variety of polynucleotides can encode a binding peptide of the invention such as those disclosed in FIG. 1, their C-C derivatives or a homologous motif binding peptide including a repeatable motif as illustrated in Table 2. The present invention encompasses all such polynucleotides.

A polynucleotide which encodes a binding peptide of the invention may be obtained by standard procedures known in the art, for example, by chemical synthesis, by PCR and by direct isolation and amplification.

D. Phenol Oxidizing Enzymes

In one embodiment the phenol oxidizing enzyme of the invention is a fungal phenol oxidizing enzyme. Phenol oxidizing enzymes are known to be produced by a wide variety of fungi and include but are not limited to species of the genii Aspergillus, Neurospora, Podospora, Botrytis, Pleurotus, Fomes, Coprinus, Phlebia, Trametes, Polyporus, Rhizoctonia, Bipolaris, Curvularia, Amerosporium, Lentinus, Myrothecium, Chaetomium, Humicola, Trichoderma, Myceliophthora, Scytalidium and Stachybotrys.

Preferred phenol oxidizing enzymes and particularly laccases are derived from Stachybotrys including S. chartarum, S. parvispora, S. kampalensis, S. theobromae, S. bisbyi, S. cylindrospora, S. dichroa, S. oenanthes and S. nilagerica; Myceliophthora including M. thermophilum; Coprinus including C. cinereus; Polyporus including P. pinsitus; Rhizoctonia including R. solani; Bipolaris including B. spicifera; Curvularia including C. pallescens; Amerosporium including A. atrum; and Scytalidium including S. thermophilum.

Many of the phenol oxidizing enzymes useful according to the invention may be obtained or produced from phenol oxidizing producing microorganisms in publicly available databases. Illustrative is Stachybotrys strains (such as S. parvispora MUCL 38996 and S. chartarum MUCL 38898). These microorganisms may be grown under aerobic conditions in nutrient medium containing assimilable carbon and nitrogen together with other essential nutrients. The medium can be composed in accordance with principles well-known in the art.

During cultivation, the phenol oxidizing enzyme producing strains secrete the enzyme extracellularly. This permits the isolation and purification (recovery) of the enzyme to be achieved by, for example, separation of cell mass from a culture broth (e.g. by filtration or centrifugation). The resulting cell-free culture broth can be used as such or, if desired, may first be concentrated (e.g. ultrafiltration). If desired, the phenol oxidizing enzyme can then be separated from the cell-free broth and purified to the desired degree by conventional methods, e.g. by column chromatography.

The phenol oxidizing enzymes according to the present invention may be isolated and purified from the culture broth into which they are extracellularly secreted by concentration of the supernatant of the host culture, followed by hydrophobic interaction chromatography or anion exchange chromatography.

Numerous references are available on suitable phenol oxidizing enzymes which may be combined or derivatized with the binding peptides of the invention, and reference is made to WO 98/38286; WO 99/49020; WO 00/37654; WO 01/21809; and U.S. Pat. No. 6,168,936;

The phenol oxidizing enzyme which comprises the binding enzyme-peptide complex may be a recombinant enzyme of a naturally occurring phenol oxidizing enzyme and methods for introducing mutations into phenol oxidizing enzymes encoding DNA sequences are known and reference is made to U.S. Pat. Nos. 4,760,025; 5,770,419; 5,985,818; 6,060,442; WO 98/27197 and WO 98/27198.

In an illustrative embodiment, a laccase enzyme which may be combined with a binding peptide to form a phenol oxidizing enzyme complex according to the invention is obtainable from any Stachybotrys species which produces a laccase capable of modifying the color associated with colored compounds. A preferred phenol oxidizing enzyme is Stachybotrys oxidase B having the amino acid sequence shown in SEQ ID NO: 1 and enzymatically active variants thereof. Typical variant enzymes in accordance with the invention will have at least 60% and less than 100% sequence identity to the amino acid sequence of SEQ ID NO: 1. That is at least 60% and less than 100%; at least 65% and less than 100%; at least 70% and less than 100%; at least 75% and less than 100%; at least 80% and less than 100%; at least 85% and less than 100%; at least 90% and less than 100%; at least 95% and less than 100%; and at least 97% and less than 100% sequence identity to the amino acid sequence of SEQ ID NO: 1.

The present invention encompasses laccase variants where the variant comprises a sequence that differs from that of SEQ ID NO: 1 in at least one of the following positions. 48, 67, 70, 76, 83, 98, 115, 119, 134, 171, 175, 177, 179, 188, 236, 246, 253, 254, 269, 272, 296, 302, 308, 318, 329, 331, 346, 348, 349, 365, 390, 391, 394, 404, 415, 423, 425, 428, 434, 465, 479, 481, 483, 499, 550, 562, 570, and 573 or sequence positions corresponding thereto. These amino acid position numbers refer to those assigned to the Stachybotrys oxidase B enzyme sequence presented in SEQ ID NO: 1.

Preferred variants include a sequence that differs from that of SEQ ID NO: 1 in at least one of the following positions 188, 254, 272, 346, 348, 394, and 425. One such variant includes an amino acid substitution in position 254 (the 254 variant) substituted with F, N, L, K, A, I, E, S, H, V, T, P, G or C, preferably F. In a further embodiment, the 254 variant is combined with at least one further substitution selected from the group consisting of positions 48, 67, 70, 76, 83, 98, 115, 119, 134, 171, 175, 177, 179, 188, 236, 246, 253, 269, 272, 296, 302, 308, 318, 329, 331, 346, 348, 349, 365, 390, 391, 394, 404, 415, 423, 425, 428, 434, 465, 479, 481, 483, 499, 550, 562, 570, and 573. Preferably the additional substituted positions are selected from 76, 188, 272, 302, 346, 348, 394 and 425. Further preferred variants include the following amino acid substitution sets:

(a) 76/188/254/302;

(b) 76/2541302;

(c) 254/394;

(d) 254/346/348, specifically M254F/E346V/E348Q;

(e) 188/254/346/348/394; and

(f) 171/179/188/254/346/348/394.

Still other preferred variants of SEQ ID NO: 1 include the substitution of amino acid residues at positions 394/425, specifically D394NN425M. This variant may further include an amino acid substitution in at least one of the positions 76, 254 and 302.

Yet another preferred variant of SEQ ID NO: 1 includes an amino acid substitution in position 272, and additionally a substitution of amino acid position 272 combined with a substitution at position 254, specifically M254F/S272L.

Polynucleotides encoding a phenol oxidizing enzyme and specifically a laccase, may be obtained by standard procedures known in the art for example, cloned DNA (e.g. a DNA “library”), by chemical synthesis, by cDNA cloning, by PCR or by the cloning of genomic DNA or fragments thereof, purified from a desired cell, such as a Stachybotrys species. Nucleic acid sequences derived from genomic DNA may contain regulatory regions in addition to coding regions. These methods are well known and reference is made to Sambrook et al., 1989, Molecular cloning, A Laboratory Manual, 2d Ed. Cold Spring Harbor Laboratory Press, Cold Spring Harbor, N.Y.; Benton and Davies, 1977, Science 196: 180; Grunstein and Hogness 1975, Proc. Natl. Acad. Sci. USA 72:3961; and U.S. Pat. Nos. 4,683,202 and 6,168,936. In one embodiment, preferred polynucleotides encode the laccase as illustrated in SEQ ID NO: 1.

E. Making the Phenol Oxidizing Enzyme-peptide Complex

The phenol oxidizing enzyme-peptide complex (also referred to as the derivatized phenol oxidizing enzyme) may be constructed by methods well known in the art including PCR. The binding peptide may be inserted into a phenol-oxidizing enzyme, may replace an internal loop or turn, and may be fused to the carbon or nitrogen terminus of the enzyme. In a preferred embodiment the binding peptide is fused to the carbon terminus.

F. Expression Systems

The present invention provides host cells, expression methods and systems for the production of the phenol oxidizing enzyme-peptide complex in host microorganisms, such as fungus, yeast and bacteria.

Molecular biology techniques are disclosed in Sambrook et al., Molecular Biology Cloning: A Laboratory Manual, Second Edition (1989) Cold Spring Harbor Laboratory Press, Cold Spring Harbor, N.Y. (1989). A polynucleotide encoding a phenol oxidizing enzyme-peptide complex is obtained and transformed into a host cell using appropriate vectors. A variety of vectors and transformation and expression cassettes suitable for the cloning, transformation and expression in fungus, yeast, plants and bacteria are known by those of skill in the art.

Typically, the vector or cassette contains sequences directing transcription and translation of the phenol-oxidizing enzyme-peptide complex, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. These control regions may be derived from genes homologous or heterologous to the host as long as the control region selected is able to function in the host cell.

Initiation control regions or promoters, which are useful to drive expression of the phenol oxidizing enzymes in a host cell are known to those skilled in the art. Virtually any promoter capable of driving these phenol oxidizing enzymes is suitable for the present invention. Nucleic acid encoding the phenol oxidizing enzyme is linked operably through initiation codons to selected expression control regions for effective expression of the oxidative or reducing enzymes. Once suitable cassettes are constructed they are used to transform the host cell.

Suitable hosts include fungus, yeast, plants and bacteria. In one embodiment the host cell is a filamentous fungus including Aspergillus species, Trichoderma species and Mucor species. In a further embodiment, the fungus includes Trichoderma reesei, Aspergillus niger and Aspergillus oryzae. In yet another embodiment, the host cell is a yeast which includes Saccharomyces, Pichia, Hansenula, Schizosaccharomyces, Kluyveromyces and Yarrowia species. In yet another embodiment the host cell is a gram positive bacteria such as a Bacillus species or a gram negative bacteria such as a Escherichia species

General transformation procedures are taught in Current Protocols In Molecular Biology (vol. 1, edited by Ausubel et al., John Wiley & Sons, Inc. 1987, Chapter 9) and include calcium phosphate methods, transformation using PEG and electroporation. For Aspergillus and Trichoderma, PEG and Calcium mediated protoplast transformation can be used (Finkelstein, DB 1992 Transformation. In Biotechnology of Filamentous Fungi. Technology and Products (eds. by Finkelstein & Bill) 113-156. Electroporation of protoplast is disclosed in Finkelestein, DB 1992 Transformation. In Biotechnology of Filamentous Fungi. Technology and Products (eds. by Finkelstein & Bill) 113-156. Microprojection bombardment on conidia is described in Fungaro et al. (1995) Transformation of Aspergillus nidulans by microprojection bombardment on intact conidia. FEMS Microbiology Letters 125 293-298. Agrobacterium mediated transformation is disclosed in Groot et al. (1998) Agrobacterium tumefaciens-mediated transformation of filamentous fungi. Nature Biotechnology 16 839-842. For transformation of Saccharomyces, lithium acetate mediated transformation and PEG and calcium mediated protoplast transformation as well as electroporation techniques are known by those of skill in the art.

As discussed above for the production of phenol oxidizing enzymes, the phenol oxidizing enzyme complex may be produced by cultivation of a host cell which includes a polynucleotide encoding the phenol oxidizing peptide complex under aerobic conditions in nutrient media containing assimiable carbon and nitrogen together with other essential nutrient. These conditions are well known in the art.

Host cells that contain the coding sequence for a phenol oxidizing enzyme-peptide complex of the present invention and express the phenol-oxidizing enzyme may be identified by a variety of procedures known to those of skill in the art. These procedures include, but are not limited to, DNA-DNA or DNA-RNA hybridization and protein bioassay or immunoassay techniques which include membrane-based, solution-based, or chip-based technologies for the detection and/or quantification of the nucleic acid or protein.

Once a phenol oxidizing enzyme-peptide complex is encoded the derivatized enzyme may be isolated and purified from the host cell by well-known techniques such as, cell separation and concentration of the cell free broth by ultrafiltration, ammonium sulfate fractionation, purification by gel filtration, ion exchange or hydrophobic interaction chromatography, PEG extraction and crystallization.

One example of purification includes small-scale purification (e.g., less than 1 g) of derivatized enzyme using hydrophobic interaction chromatography. Samples may be filtered and loaded onto a column containing 20HP2 resin (Perceptives Biosystems), hooked up to a BioCad workstation (Perceptives Biosystems). The column may be washed with ammonium sulfate in buffer. Elution of the derivatized phenol oxidizing enzyme activity can be performed using a salt gradient ranging from 35% to 0% of a 3M ammonium sulfate solution in 30 mM Mes Bis Tris Propane buffer at pH 5.4. The fractions enriched in the derivatized phenol oxidizing enzyme activity can be monitored using UV absorbance at 280 nm and a qualitative ABTS activity assay. The samples can be pooled, concentrated and diafiltered against water. Derivatized samples purified according to this method are estimated to be at least about 70% pure.

F. Applications

1. Enzyme and Detergent Compositions

A phenol oxidizing enzyme-peptide complex of the present invention may be used to produce, for example, enzymatic compositions for use in detergent or cleaning compositions; such as for removing food stains on fabrics; and in textiles, that is in the treatment, processing, finishing, polishing, or production of fibers.

Enzymatic compositions may also comprise additional components, such as for example, for formulation or as performance enhancers. For example, detergent composition may comprise, in addition to the phenol oxidizing enzyme-peptide complex, conventional detergent ingredients such as surfactants, builders and further enzymes such as, for example, proteases, amylases, lipases, cutinases, cellulases or peroxidases (U.S. Pat. No. 4,689,297). Other ingredients include enhancers, stabilizing agents, bactericides, optical brighteners and perfumes. The enzymatic compositions may take any suitable physical form, such as a powder, an aqueous or non-aqueous liquid, a paste or a gel.

A phenol-oxidizing enzyme-peptide complex of the present invention can act to modify the color associated with dyes or colored compounds in the presence or absence of enhancers depending upon the characteristics of the compound. If a compound is able to act as a direct substrate for the phenol oxidizing enzyme, the phenol oxidizing enzyme will modify the color associated with a dye or colored compound in the absence of an enhancer, although an enhancer may still be preferred for optimum phenol oxidizing enzyme activity. For other colored compounds unable to act as a direct substrate for the phenol oxidizing enzyme or not directly accessible to the phenol oxidizing enzyme, an enhancer may be required for optimum phenol oxidizing enzyme activity and modification of the color.

Enhancers are described in for example WO 95/01426, WO 96/06930, and WO 97/11217. Enhancers include but are not limited to phenothiazine-10-propionic acid (PTP), 10-methylphenothiazine (MPT), phenoxazine-10-propionic acid (PPO), 10-methylphenoxazine (MPO), 10-ethylphenothiazine-4-carboxylic acid (EPC) acetosyringone, syringaldehyde, methylsyringate, 2,2′-azino-bis(3-ethylbenzothiazoline-6-sulfonate (ABTS), 2,6 dimethoxyphenol (2,6-DMP), and guaiacol (2-methoxyphenol).

2. Other Applications

The phenol oxidizing enzyme-peptide complexes may also be useful in applications other than enzyme and detergent compositions for stain removal. In one preferred embodiment the peptides according to the invention bind preferentially to carotenoid and xanthophyll chromophores. Therefore other applications may include personal care applications, for example in skin cosmetics as skin tanners, food industry applications, for example as fruit ripening agents or in diagnostic uses, such as in pharmaceutical applications, for example to localize presence of carotenoids in tissue.

Having thus described the binding peptides and the phenol oxidizing enzyme-peptide complexes of the present invention, the following examples are now presented for the purposes of illustration and are neither meant to be, nor should they be, read as being restrictive. Dilutions, quantities, etc. which are expressed herein in terms of percentages are, unless otherwise specified, percentages given in terms of per cent weight per volume (w/v). As used herein, dilutions, quantities, etc., which are expressed in terms of % (v/v), refer to percentage in terms of volume per volume. Temperatures referred to herein are given in degrees centigrade (C).

The manner and method of carrying out the present invention may be more fully understood by those of skill in the art by reference to the following examples, which examples are not intended in any manner to limit the scope of the present invention or of the claims directed thereto. All references and patent publications referred to herein are hereby incorporated by reference.

### EXPERIMENTAL

### EXAMPLE 1

Selection of the Binding Deptides on Stained Cotton.

While a number of selection techniques may be used to screen for binding peptides, the majority of the binding peptides according to the invention were selected according to the method described herein below.

10 microliters of a commercially (New England Biolabs) available phage display library either a cyclic 7-mer (at 2.10E13 pfu/ml) or a linear 12-mer (at 4.10E12 pfu/ml) were pre-incubated with a cotton swatch in a pre-blocked and washed 96 well plate in the presence of a 150 μl TBS solution (at 2.10E-5 g/l for the cyclic 7-mer, 2.10E-3 g/l for the linear 12-mer) of detergent, pH 10 for 20 minutes using gentle shaking. The solution was pipetted off and added to a second cotton swatch for 20 minutes under gentle shaking. This process was repeated a third time. The solution was pipetted off and added to a tomato (Textile Innovators, NC) or paprika (Test Fabrics, PA) stained swatch for 60 minutes under gentle agitation. The solution was drawn off and discarded. The stained swatch was washed 5× for 5 minutes each with 200 μl of TBST (TBS containing 0.1% Tween 20). The swatch was transferred to an empty well using sterile tips, washed as described above, and transferred to another empty well. 15 μl of a glycine 0.2M solution pH 2.2 was added to the stained swatch and the plate was shaken for less than 10 minutes. This solution was neutralized by the addition of 100 μl of a Tris HCL 1M solution, pH 9.1 for 10 minutes. The solution, which constitutes the acid eluted peptide population was pipetted off and stored at 4° C. until further use.

4×20 μl of the acid eluted phage peptide population was used to infect 4×400 μl E. coli (New England BioLabs) grown to an OD at 610 nm of 0.3 to 0.65 from a 100× dilution in LB of an overnight culture. The cells were plated on 4×140 mm LB plates in the presence of IPTG (Sigma) (40 μl at 20 mg/ml per plate) and Xgal (Sigma) (40 μl at 40 mg/ml of DMF per plate) added to 5 mls of melted top agarose, and left to incubate overnight at 37° C. The 4 plates were scraped with a sterile glass microscope slide and the scrapings were pushed through an 18.5 gage needle of a 60 ml syringe into a sterile conical tube; 50 ml of TBS was added to the tube and the capped tube was left to shake on a rocker at room temperature for at least 14 hrs. The contents of the tube were centrifuged at 10,000 rpm for 30 minutes in sterile Oakridge tubes at 4° C. The supernatant was collected and the phage precipitated by adding 1/6 volume of a 20% polyethylene glycol (PEG)/2.5 M NaCl solution. This was left to incubate at 4° C. for at least 4 hours and preferably overnight. The solution was then spun at 10,000 rpm for 30 minutes at 4° C. and the supernatant discarded. The pellet was resuspended in 1 ml of TBS and transferred to a sterile Eppendorff tube. The phage was reprecipitated with 1/6 volume of a 20% PEG/2.5 M NaCl solution with incubation on ice for at least 1 hour. This was followed by another centrifugation at 10,000 rpm for 10 min at 4° C. The supernatant was discarded, the tube re-spun briefly, and residual supernatant removed. The pellet was resuspended in 200 μl TBS/0.02% NaN₃, spun to remove insoluble material and transferred.

The amplified phage peptide populations from the first round of deselection on cotton/selection of stained cotton swatches were submitted to another round of deselection and selection as described above. For the cyclic 7-mer peptide library 2.10E-4 g/l TBS was used, and for the linear 12-mer peptide library 2.10E-2 g/l TBS was used. After acid elution and amplification of the phage, a third round of biopanning was performed. The third round used 2.10E-3 g/l TBS of detergent for the cyclic 7-mer phage peptides and 2.10E-1 g/l TBS for the linear 12-mer phage peptides. After acid elution and amplification a fourth round of biopanning was used and 2 g/l of detergent dissolved in water in one experiment and TBS in another were used for both types of phage peptides. The phage peptides were acid eluted and amplified from the fourth round of biopanning and selected in a fifth round of biopanning wherein the Tween 20 concentration was increased from 0.1% to 0.8% in the wash conditions. Additionally a round of selection on tomato and paprika was performed using the phage peptides from the third round as described above. In this fourth round 2 g/l of detergent in water in the wash conditions was used.

### EXAMPLE 2

Sequencing of the Phase Peptide Population:

225 μl of a 1/100 dilution of an overnight culture of E. coli cells in LB broth were incubated with phage plaques using sterile toothpicks in a sterile 96-well V-bottom plate. A replica plate was made for glycerol stocks of the phage peptides. The plates were covered with porous Qiagen plate sealers and shaken for 4 hours at 37° C. at 280 rpm in a humidified shaker box and then spun at 4000 rpm for 30 min at 4° C. 160 μl of the phage peptides supernatant was transferred to another 96-well V-bottom plate containing 64 μl of 20% PEG/2.5 M NaCl. The plates were left to shake for 5 minutes and then left to stand for 10 minutes. The glycerol stock plate was prepared by adding 100 μl phage supernatant to 150 μl 75% glycerol solution in a sterile 96 well plate which was then sealed with parafilm, labeled, and stored at −70° C. until further use.

The PEG precipitated phage plate was centrifuged at 4000 rpm for 20 minutes at 4° C. The plate was inverted rapidly to remove excess PEG/NaCl and left upside down on a clean paper towel to drain residual fluid. 60 μl of iodide salt solution (10 mM Tris.HCl, pH 8.0, 1 mM EDTA, 4 M NaI) were added to each well and the phage pellets thoroughly resuspended by shaking the plate vigorously for 5 minutes. 150 μl of 100% EtOH were added and the plate was spun at 4000 rpm for 20 minutes at 4° C., the supernatants discarded and the plate blotted. The pellets were washed with 225 μl of 70% EtOH without disturbing the pellets; the plate was inverted and left to air-dry for at least 30 minutes. The pellets were resuspended in 30 μl of Tris.HCl 10 mM, pH 8.5 buffer by shaking the plate for 30 minutes at full speed. 1 μl of g96 reverse primer (obtained from New England BioLabs, 3.4 pmole per tube) was added to 11 μl of DNA pellet sample and the contents submitted for sequencing on a ABI Applied Biosystem 373XL.

FIG. 1 (SEQ ID NOS: 2-433) illustrates the amino acid sequence of numerous binding peptides determined according to the method herein described. Various repeatable motifs were found in these peptides by visual and computer analyzed methods and repeatable motifs of 3 to 5 amino acid residues are reported in Table 2 along with some representative sequence identifiers for binding peptides illustrated in FIG. 1 which include the repeatable motif.

### EXAMPLE 3

Sites for Attachment and Substitution of Binding Peptides.

A. Insertion into the C-Terminus of Stachybotrys Oxidase B:

Primer Design

Reverse Primer:

| | |
|---|---|
| 3′ | |
| ACTACGGCGACTCCTCNNNNNNNNNNNNNNNNNN | (SEQ ID NO:434) |

5′
NNNATTAGATCTGGGG

wherein the 16 bp overlap with the polynucleotide sequence encoding SEQ ID NO: 1 is underlined, the section of N's symbolizes the polynucleotide encoding a binding peptide of the invention; the ATT stop codon is in bold letters, and the Xba I restriction site is doubled underlined. In a specific example the polynucleotide TTCCGGAGTCGAGGACGAAAC (SEQ ID NO: 435) encoding binding peptide KASAPAL (SEQ ID NO: 24) was added to the C-terminus.

Forward Primer HM 358 was used for all PCR reactions.

5′ AAGGATCCATCAACATGATCAGCCAAG 3′ (SEQ ID NO: 436)

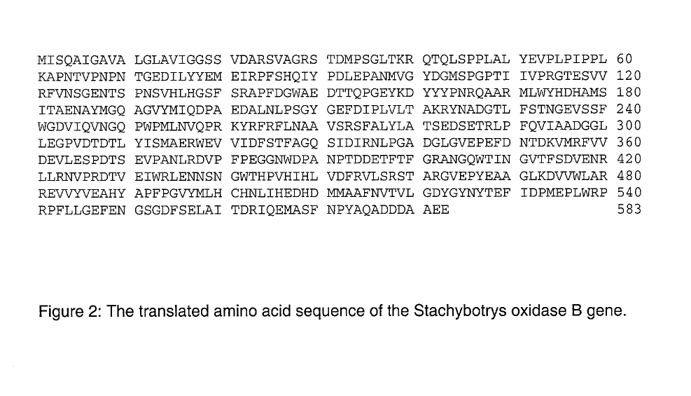

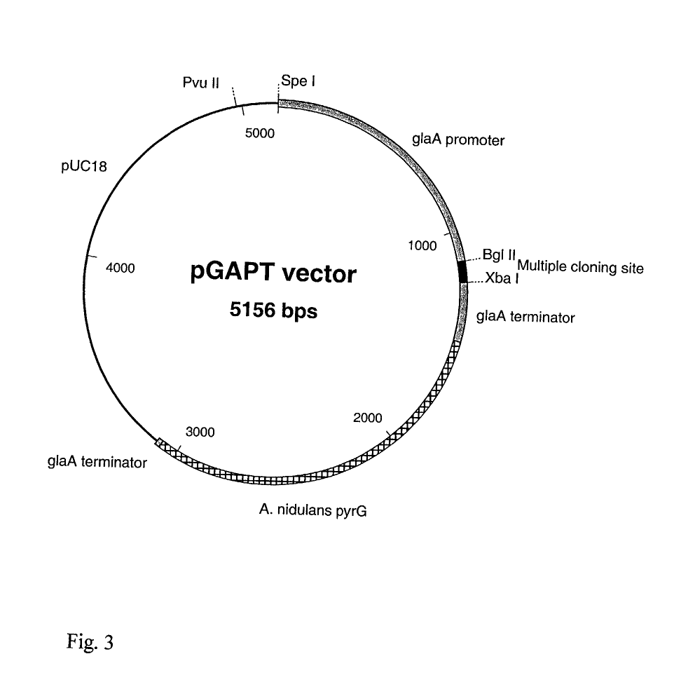

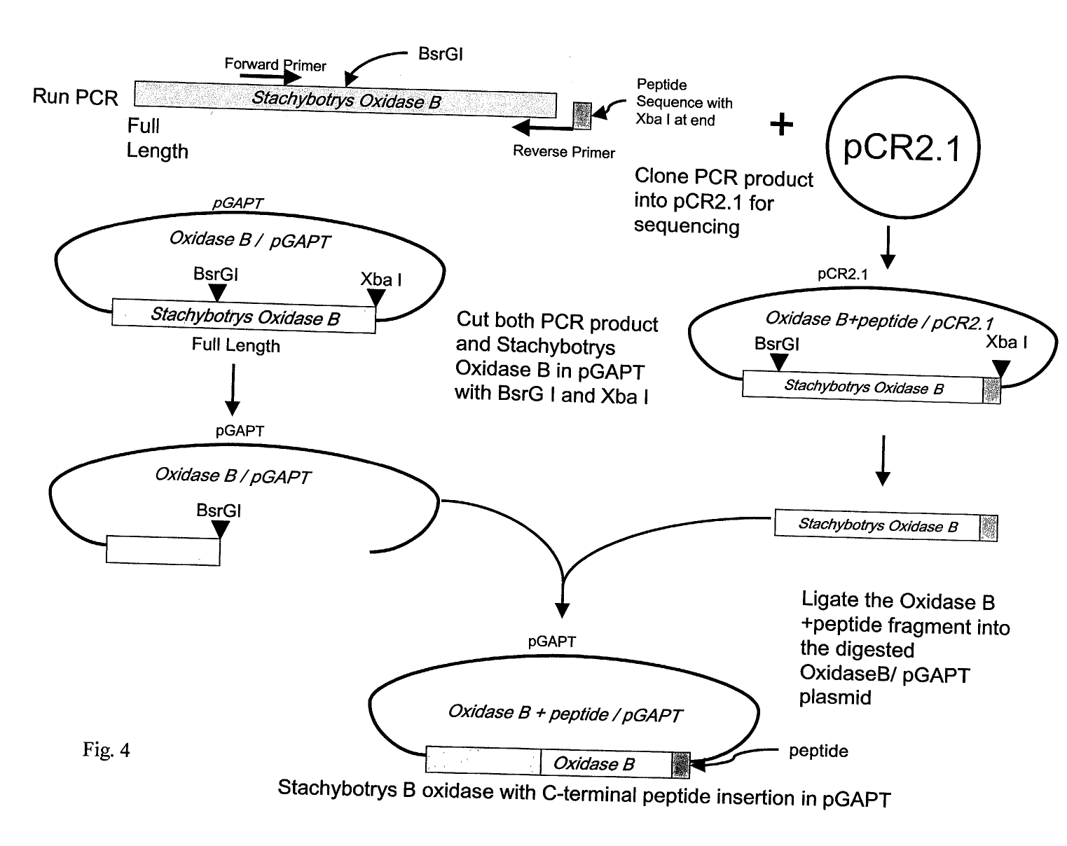

Various 7-mer, 7-mer with cysteines and 12-mer binding peptides illustrated in FIG. 1 were inserted into the C-terminus of Stachybotrys phenol oxidase B (FIG. 2), and reference is made to FIGS. 3 and 4. Primers were designed as described above. The insertion location was just past E583 at the C-terminus of Stachybotrys phenol oxidase B. (Also see FIG. 1 of WO 01/21809). PCR was used for insertion of sequences. 3′-5′ peptide primers were designed specifically for the reaction. Ten microliters of diluted DNA were added to a mixture which contained 0.2 mM of each nucleotide (A, G, C and T), 1×reaction buffer, 1.7 microgram of peptide insertion reverse primer and the common forward primer in a 100 μl reaction in an eppendorf tube. After a 5 minute incubation at 100° C., 2.5 units of Taq DNA polymerase was added to the reaction mix. The PCR reaction was begun at 95° C. for 1 minute, followed by primer annealing to the template at 50° C. for 1 minute and extension was done at 72° C. for 1 minute. The cycle was repeated 30 times with an additional cycle extension at 68° C. for 7 minutes, The final PCR product size was 975 bp. Stachybotrys phenol oxidase B (SEQ ID NO: 1) and specific variants thereof M254F and M254F/E346V/E348Q were used as the template for PCR. The fragment was purified with the Qiagen PCR Purification kit. After purification, the fragment was digested with the restriction enzymes BsrG I and Xba I in a joint digestion. The Xba I site was introduced in the PCR reaction. The BsrG I site was located 75 bp downstream from the beginning of the PCR product at 1312. Also digested was the nonderivatized Stachybotrys B phenol oxidase/pGAPT (without a binding peptide insertion or substitution) in the pGAPT expression vector. Stachybotrys B phenol oxidase/pGAPT was also digested with BsrG I and XbaI in order to facilitate cloning of the PCR product into Stachybotrys B phenol oxidase. The digested PCR product was ethanol precipitated to clean and purify the fragment and the digested Stachybotrys B phenol oxidase/pGAPT sample was run on a gel to separate the two fragments produced by the reaction (BsrG I and Xba I are both single cutters in Stachybotrys B phenol oxidase/pGAPT). The larger of the fragments was 5.8 kb while the smaller of the fragments was 945 bp long. The 5.8 kb fragment was excised from the gel and purified using the Bio 101 Geneclean III kit. The purified PCR fragment and 5.8 kb Stachybotrys B phenol oxidase/pGAPT fragment were then ligated together. The ligated DNA was then transformed into Invitrogen Toplo E.coli. Individual colonies from the transformation plate were picked and cultured in LB+50 ppm carb. overnight. The plasmid DNA was then isolated and purified using the Qiagen Miniprep kit. The isolated DNA was sequenced to check if peptide sequences were inserted, in the correct location and were the correct sequence. Sequencing was also done earlier in the process after PCR to check insertion of peptide sequences. After PCR was run, the products were ligated into the Invitrogen pCR2.1 cloning vector and sequenced. Samples were then transformed into Aspergillus niger.

The above procedure was repeated with 92 different binding peptides of the invention. The corresponding 3′-5′ primers were mixed together and PCR was run with that primer mixture and the 5′-3′ primer.

B. Insertion and Substitution into Stachybotrys Oxidase B and Variants thereof:

(1) Primer Design (7-mer, Insertion)

| 5′ | |
|---|---|
| NNNNNNNNNNNNNNNNNNNNNCCTTTCCCCGAGG | (SEQ ID NO:447) |
| 3′ | |
| GCGG | |
| 3′ | |
| GGTTGGAGGCTCTACAANNNNNNNNNNNNNNNNN | (SEQ ID NO:448) |
| 5′ | |
| NNNN | |

wherein the overlap with the polynucleotide sequence encoding SEQ ID NO: 1 is underlined and the section of N's indicates the binding peptide coding region.

(2) Primer Design (7-mer, Substitution)

| 5′ | |
|---|---|
| GAGGGCGGCAACNNNNNNNNNNNNNNNNNNNNNG | (SEQ ID NO:449) |
| 3′ | |
| ATGACGAGACTTTCACC | |

3′

| | |
|---|---|
| AAGGGGCTCCCGCCGTTGNNNNNNNNNNNNNNNN | (SEQ ID NO:450) |

5′
NNNNNCTACTGCTCTG

wherein the overlap with the polynucleotide sequence encoding SEQ ID NO: 1 is underlined and the section of N's indicates the binding peptide coding region. In a specific example the primers for insertion of binding peptide sequence SSLNATK (SEQ ID NO: 4) are:

Forward Primer

| Forward Primer | |
|---|---|
| 5′ | |
| TCCCTTCTTAACGCTACTAAGACCTTCTCGGATG | (SEQ ID NO:451) |
| 3′ | |
| TCGAG | |
| Reverse Primer | |
| 3′ | |
| CCTGTTAGTTGCCTCAAAGGGAAGAATTGCGATG | (SEQ ID NO:452) |
| 5′ | |
| ATTC | |

In a specific example the primers for substitution of binding peptide sequence SSLNATK (SEQ ID NO: 4) are:

Forward Primer

| Forward Primer | |
|---|---|
| 5′ | |
| GAGGGCGGCAACTCCCTTCTTAACGCTACTAAGG | (SEQ ID NO:453) |
| 3′ | |
| ATGACGAGACTTTCACC | |
| Reverse Primer | |
| 3′ | |
| AAGGGGCTCCCGCCGTTGAGGGAAGAATTGCGAT | (SEQ ID NO:454) |
| 5′ | |
| GATTCCTACTGCTCTG | |

Three sites within Stachybotrys B phenol oxidase (SEQ ID NO: 1) were chosen for 7-mer and 12-mer peptide insertion: site A located between V379 and P380; site B located between V412 and T413; and site C located between L422 and R423. The amino acid sequence W387, D388, P389, A390, N391, P392, and T393 was chosen for the site of 7-mer peptide substitution. All of the peptides were inserted into the Stachybotrys B phenol oxidase sequence using mutagenesis PCR. The PCR reaction allowed the peptide coding sequence to be inserted/substituted into the Stachybotrys B phenol oxidase/pGAPT plasmid without the need for cloning procedures such as restriction digest and ligation. After PCR was run, the plasmid was sequenced to verify the insertion/substitution reaction. PCR was run with the Stachybotrys B phenol oxidase/pGAPT full plasmid as the template for the reaction. The DNA was diluted 1:10 to 74.4 ng/ul and either 1.8 or 3.7 ul was added to the reaction, which also contained 0.2 mM of each nucleotide, 1×reaction buffer, and 182 nanograms of primer. 2.5 units of Stratagene PFU Turbo polymerase was added to the reaction mixture. The PCR reaction was done at 95° C. for 35 seconds followed by primer annealing to the template at 55° C. for 1 minute 5 seconds. Extension was done at 68° C. for 15 minutes and 30 seconds. The cycle was repeated 16 times. After the full length plasmid PCR product was purified with the Qiagen PCR purification kit, samples were sequenced for confirmation of peptide insertion/substitution. Successfully inserted or substituted peptides sequences in pGAPT plasmid were transformed into Aspergillus niger for expression.

### EXAMPLE 4

Expression of Laccase-peptide Complexes by Aspergillus Host Cells

The DNA fragment containing nucleic acid encoding the Stachybotrys phenol oxidase B (SEQ ID NO: 1) with the introduced binding peptide followed by a stop codon and an Xba I site was isolated by PCR. The PCR fragment was cloned into the plasmid vector pCR2.1 and subjected to nucleic acid sequencing for verification. The DNA fragment was cloned into the BsrG I to Xba I site to create a plasmid pGAPT (see FIGS. 3 and 4). The pGAPT plasmid was co-transformed with a pHELP1 plasmid (Current Genetics 24:520-524 (1993)) in Aspergillus niger to generate transformants containing the replicating plasmid. Transformants were selected on plates without uridine and grown for 3 days. Spores from the transformants were resuspended in 200 μl of Robosoy media in a 96-well plate and grown for 30° C. for 4 days. Samples were filtered and analyzed.

### EXAMPLE 5

Purification of Laccase from Fermentation Cultures.

Samples obtained as described in Example 4 were purified using small-scale hydrophobic interaction chromatography. Fermentation cultures were filtered over miracloth to separate the cells from the broth. The filtrate was further filtered through a 0.2 μm Steritop (GP) filter unit. The material was loaded onto a column containing the HIC resin 20 HP2 (Perkin Elmer), connected to a BioCad/Sprint workstation (Perkin Elmer) after the resin had been equilibrated with 1.05 M ammonium sulfate in 30 mM Mes, Bis-tris Propane, pH 5.4 buffer. After washing the column to an ammonium sulfate concentration of 0.75M, the enzyme-peptide complex was eluted using ammonium sulfate gradient going from 0.75M to 0.0M over 5CVs. All fractions were quickly checked for ABTS activity using a qualitative assay in which 50 μL of fraction were added to 100 μL of an ABTS solution (4.5 mM)) in a 96 well titer plate; apparition of a teal green color in less than 10 sec indicated the enriched presence of laccase. In parallel, the fractions were loaded onto a SDS gel (Nu PAGE, 4-12%, Invitrogen) to assess the purity of the fractions. The enriched and purified fractions were pooled, concentrated using a Pellicon XL unit (MWCO: 8000 Da, Millipore), further concentrated and diafiltered against Milli-Q water using YM-10 centripreps until the permeate reached a conductivity of around 5 μS. The enriched fraction was then frozen at −70° C. in 1 ml aliquots until further use. The purity of the enzyme obtained as described was often superior to 80-90%.

### EXAMPLE 6

Preferential Binding of the Tomato Binding Peptide YGYLPSR (SEQ ID NO: 16)

The following stock solutions were prepared:

2 g/L Lever “Multi Acao” detergent

10 mM NiSO4

2 mM STP #1 (GGHGGYGYLPSR) (SEQ ID NO: 455)

2 mM STP #2 (GGHGGCYGYLPSRC) (SEQ ID NO:456)

10 mM GGH

OPD (o-Phenylene Diamine, Sigma P-8287 10 mg tablet/22.5 mL buffer (50 mM HEPES, pH 8.0)

100 mM H2O2 stock

Appropriate amounts of NiSO4 and Ni-STP #1 (GGHGGYGYLPSR) stock solutions were mixed to prepare 0.125-1.0 mM Ni-STP#1 solutions. The resulting solutions were mixed for at least 10 minutes before using to form the Ni-peptide complex. Appropriate amounts of NiSO4 and Ni-STP #2 (GGHGGCYGYLPSRC) stock solutions were mixed to prepare 0.125-1.0 mM Ni-STP#2 solutions. The resulting solutions were mixed for at least 10 minutes before using to form the Ni-peptide complex. Appropriate amounts of NiSO4 and GGH stock solutions were mixed to prepare 0.125-1.0 mM Ni-GGH solutions. The resulting solutions were mixed for at least 10 minutes before using to form the Ni-peptide complex.

An appropriate number of tomato stained cotton swatches and unstained cotton swatches were added to a 96 well plate. 100 μL nickel peptide stock solutions were added to the 96 well plate with the swatches and the resulting mixture incubated for 90 minutes at room temperature with gentle rocking. After incubation, the solution was removed with suction and each swatch rinsed 2 times in 200 μL dH₂O by shaking for 3 minutes. 200 μL OPD solution and 50 μL of H₂O₂ solution was added to each well and the plate place on a shaker at moderate speed. The mixture was allowed to incubate overnight and then 200 μL was transferred from each well to a new 96 well plate. Absorbance was read at 430 nm.

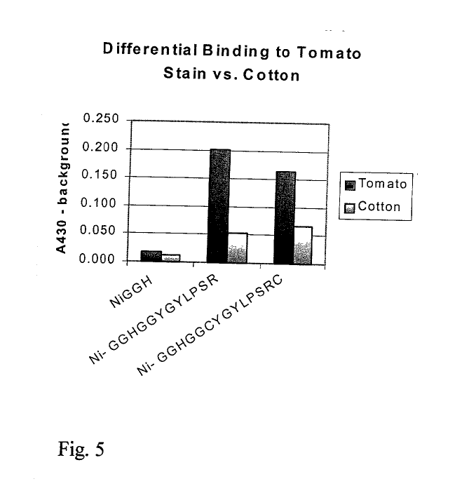

FIG. 5 shows a comparison of binding to tomato stain vs. unsoiled cotton from a starting concentration of 0.5 mM Ni-peptide. The NiGGH values were adjusted for higher activity by dividing by 3; to bring the absorbance values in line with the other Ni-peptide values and provide an equal basis of comparison. The plot shows STP #1, Ni-SEQ ID NO: 455, binds to tomato stain about 4× more than to cotton, STP #2, Ni-SEQ ID NO: 456, binds to tomato stain about 3× more than to cotton, and NiGGH shows no preferential binding.

### EXAMPLE 7

Laccase-peptide Complex Binding.

Four samples were used to test the binding ability and other properties of 3 laccase-peptide complexes according to the invention. As discussed above the laccase-peptide complex comprised a binding peptide that was attached to the laccase at the C-terminus. The samples included (a) SEQ ID NO: 1-IERSAPATAPPP (SEQ ID NO: 92); (b) SEQ ID NO: 1-the C-C derivative of KASAPAL (SEQ ID NO: 24); (c) SEQ ID NO: 1-KASAPAL (SEQ ID NO: 24); and nonderivatized laccase SEQ ID NO: 1.

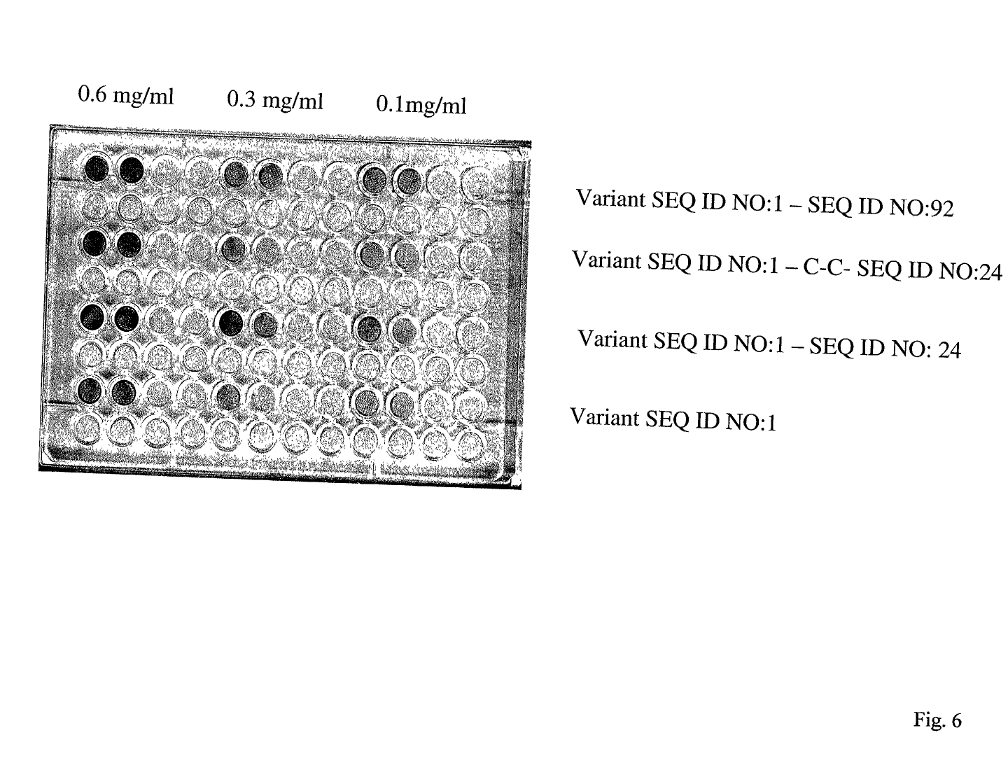

A 96 well plate was filled with cotton swatches stained with tomato (Textile Innovators). 90 μL of 83.5 mM sodium carbonate, pH 10 buffer were added to the swatches. 50 μL of purified enzyme dilutions, protein concentrations of 0.6 mg/ml, 0.3 mg/ml and 0.1 mg/ml, were added and the plate was left to incubate at room temperature for an hour using mild shaking. The solution was pipetted off and the swatches rinsed with 150 μL of MilliQ water using strong agitation for 5 min. The rinse pipetted off; the swatches received 150 μL of an ABTS solution (4.5 mM in 50 mM sodium acetate, pH 5). Qualitative estimation of binding of the complex was observed and evaluated by visual determination of the dark green color caused by ABTS oxidation (FIG. 6). As observed the results indicate the superior binding on a protein basis of the laccase-peptide complex versus the original nonderivatized laccase.

Additionally a guaiacol assay and protein concentration were determined as outlined below with results represented in Table 3.

TABLE 3

| | | Av | Av | | Protein |
|---|---|---|---|---|---|
| | | Guaiacol | Guaiacol | Guaiacol | Concen- |
| | Av ABTS | pH 8.5 | pH 10.0 | Ratio | tration |
| SAMPLE | U/ml | U/ml | U/ml | 10/8.5 | Mg/ml |
| SEQ ID NO: | 16.13 | 6.375 | 8.348 | 1.31 | 0.623 |
| 1-ERSAPATA | | | | | |
| PPP (SEQ | | | | | |
| ID NO: 92) | | | | | |
| SEQ ID NO: | 18.48 | 8.462 | 11.735 | 1.39 | 1.23 |
| 1-KASAPAL | | | | | |
| (SEQ ID | | | | | |
| NO: 24) | | | | | |
| SEQ ID NO: | 21.25 | 11.119 | 14.173 | 1.28 | 0.657 |
| 1-C-SEQ ID | | | | | |
| NO:24-C | | | | | |
| SEQ ID NO: | 12.55 | 7.326 | 7.731 | 1.06 | 1.19 |
| 1 | | | | | |

The guaiacol assay is also useful for determining phenol oxidizing activity, especially at higher pH levels. The following reagents are used: 50 mM Tris-HCl buffer pH 8.5 (To make 1 L: dissolve 7.8 g of Tris-HCL in 1 L of DI water. Mix gently. Calibrate pH probes and adjust pH to 8.5. Buffer should be filter sterilized using a 0.2 um filter); 50 mM Guaiacol in Milli-Q-H₂O (To make 20 mL of 50 mM Guaiacol: dissolve 124 uL of Guaiacol (Sigma catalog number 6-5502) in Milli-Q- H₂O. Guaiacol is light sensitive; solutions containing Guaiacol should be kept away from light by shielding container. This reagent solution should be made fresh daily for quality purposes.

The reagents are combined as follows:

| Guaiacol stock solution | final [conc] |
|---|---|
| 750 μL of pH 8.5 Tris-HCl 50 mM buffer | 42 mM Tris-HCl |
| 100 μL of 50 mM Guaiacol | 5.6 mM Guaiacol |

The enzyme-peptide complex sample is diluted in water, if necessary. 750 μL of Tris-HCl buffer, 100 μL of guaiacol, and 50 μL of enzyme are added to a disposable 1.5 mL cuvette. The reaction is allowed to proceed for 30 seconds at ambient room temperature of 21° C. and a reading is taken every 2 seconds using a spectrophotometer at a lambda of 470 nm. Before the first reading, mix the reaction solution well in the cuvette.

The following calculation can be carried out:

$$\begin{matrix}Specific activity = ( ( Δ OD units \text{/} min ) / ( 0.050 mL ) ) / ( [ protein ] mg \text{/} mL ) \\ = Δ OD units \text{/} min \text{/} mg protein\end{matrix}$$

Protein concentration can be estimated, for example, using the BCA protein assay (See, e.g., Smith, P. K., et al (1985) “Measurement of protein using bicinchoninic acid.” Anal. Biochem. 150: 76-85).

In an exemplary procedure, employing the Pierce BCA Protein Assay Reagent Kit (Product Cat. 23225) (Pierce; Rockford, Ill.) [Reference: Pierce Protein Assay Reagent Kit Instructions (for protein assay)]:

1) Prepare Pierce BCA Protein kit Working Reagent (WR):

a) Mix 50 parts of Reagent A (Sodium carbonate, sodium bicarbonate, BCA detection reagent and sodium tartarate in 0.1 M NaOH) with 1 part of Reagent B (4% CuSO₄.5H₂O)

2) Prepare BSA std.s using 2 mg/mL BSA std. stock soln.

See Mfrs. Instructions (diln.s prepared in Milli-Q water)

Chill 20% TCA thoroughly:

1) 50 uL of Sample/Std.s & 50 uL of 20% TCA>mix>put on ice for 20 min.

2) Centrifuge for 10 minutes>Decant>Dry in Speed Vac

Speed Vac: Bring to speed>turn on vac.>run˜2 min.>turn vac. off>stop and remove samples

3) Resuspend in 50 uL of WR

4) Add 1 mL WR to each tube

5) Incubate at 37° for 30 minutes

6) Cool to Rm. Temp. and read at 562ₙₘ

Plot Standards and Determine Protein Concentrations:

1) Do Scatter plot on Standards

2) Determine trend line

3) Display equation and R² value:

use the equation to determine protein conc.: y=mx+b where: y=562 nm reading, and x=ug/mL

Protein determination in connection with unpurified complexes can be done by way of a different protocol; for example, the protein can be quantified via densitometry on Coomassie stained SDS gels.

### EXAMPLE 8

Binding of Laccase-YGYLPSR (SEQ ID NO: 16) to Tomato:

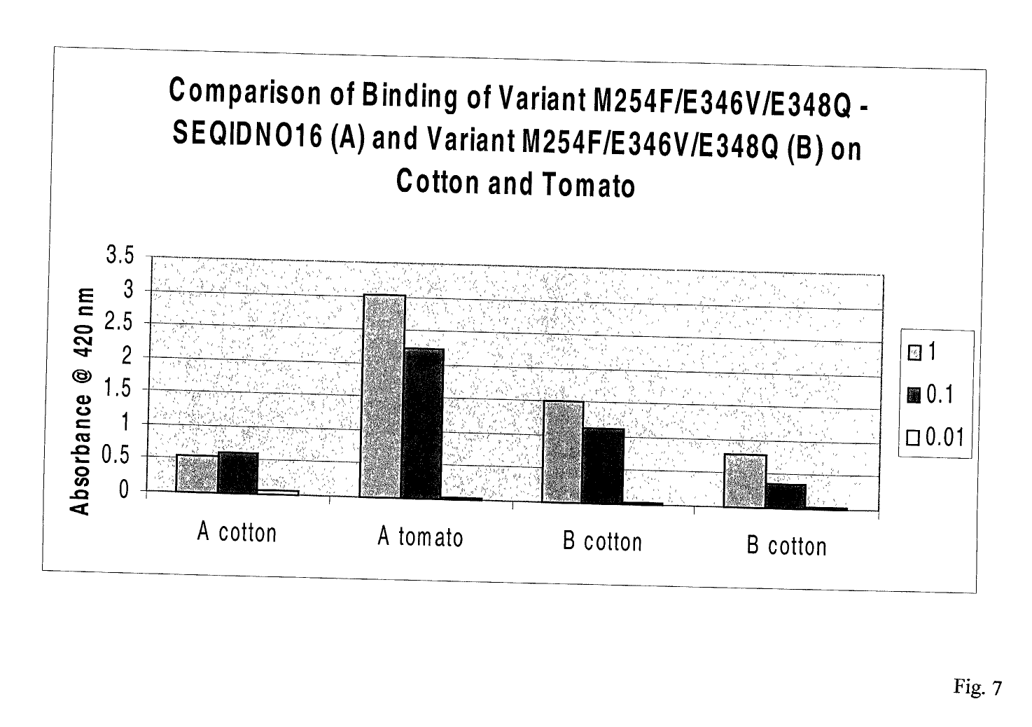

Tomato stained swatches (Textile Innovators Corp.) and non-stained cotton swatches (Textile Innovators Corp.) were placed in wells of a 96 well titer plate, previously blocked with a solution of BSA in PBS (Superblock, Pierce), for 2 days at room temperature and rinsed three times with MilliQ water (with 150 ul per well), Dilutions (100 ul) of SEQ ID NO:1, variant M254F/E346V/E348Q-YGYLPSR (SEQ ID NO: 16) or the same variant without SEQ ID NO: 16 (1 mg/ml, 0.1 mg/ml and 0.01 mg/ml) in a commercial detergent solution were added in duplicate to the non-stained cotton swatches and to the tomato stained cotton swatches. Incubation was at 1 hr at room temperature with moderate shaking. The incubation solution was pipetted off and the swatches were washed twice with 150 ul MilliQ water for 1 minute with moderate shaking. 150 ul of a 4.5 mM solution of ABTS in sodium acetate 50 mM, pH 5 buffer were added to each swatch. After 5 minutes incubation under moderate agitation 100 ul of the ABTS solutions were placed in an empty 96 well plate and the absorbance at 420 nm was read (end point assay) against blanks containing only the original ABTS substrate solution, The average absorbance (n=2) for each concentration of laccase for each type of swatch is depicted in FIG. 7. The results indicate the laccase variant combined with SEQ ID NO:16, designated as (A) bound at least 4 to 6 times greater to tomato stained swatches than to cotton swatches. The results also indicate that A bound approximately 4 to 9 times greater to tomato and about 2 times greater to cotton than the non-derivative laccase variant designated as (B).

What is claimed is:

1. A binding peptide having the amino acid sequence illustrated in any one of SEQ ID NOS: 2 through 433.

2. The binding peptide of claim 1, wherein said peptide is selected from the group consisting of SEQ ID NOS: 4, 16, 24, 77, 92, 94, 104, 105, 120, 198, 233, 237, 243, 247, 279, 293, 300, 304, 317, 340, and 428.

3. The binding peptide of claim 2, wherein said peptide is selected from the group consisting of SEQ ID NOS: 4, 16, 24, 92, 94, 104, 105, 120, 198, 233, 247, 279, 293, 300, 304, and 317.

4. The binding peptide of claim 1, further comprising a cysteine amino acid residue added to each end of the binding peptide.

5. A binding peptide comprising a repeatable motif selected from the group consisting of SAPA, TAPP, APAL, PPP, PPPP, SSPH, SSP, SSK, SPT, LPAQ, PPPL, PTPL, SPTT, PLVP, PLP, YTKP, SLH, SLLNA, SPL, SNLA, SPLTQ, TTT, AARND, AARN, ARND, LSPG, NPNN, NLAT, NTS, PHSM, PPWM, PTSP, TGGA, YLPS, YTKP, PGSL, APS, TPV, TTTS and LNAT, wherein said binding peptide has 6 to 15 amino acid residues and binds to a stain on a fabric.

6. A polynucleotide sequence encoding the binding peptide of claim 1.

7. A polynucleotide sequence encoding the binding peptide of claim 5.

8. A phenol oxidizing enzyme-peptide complex comprising a phenol oxidizing enzyme and a peptide having the amino acid sequence illustrated in any one of SEQ ID NOS: 2 through 433.

9. The phenol oxidizing enzyme-peptide complex of claim 8, wherein the binding peptide is selected from the group consisting of SEQ ID NOS: 4, 16, 24, 92, 94, 104, 105, 120, 198, 233, 247, 279, 293, 300, 304, and 317.

10. The phenol oxidizing enzyme-peptide complex of claim 8, wherein the peptide is attached to the phenol-oxidizing enzyme at the C-terminus.

11. The phenol oxidizing enzyme-peptide complex of claim 8, wherein the peptide replaces an internal amino acid sequence of the phenol-oxidizing enzyme.

12. The phenol oxidizing enzyme-peptide complex of claim 11, wherein the peptide replaces an internal amino acid sequence in an internal loop structure of the phenol oxidizing enzyme.

13. The phenol oxidizing enzyme-peptide complex of claim 8, wherein the phenol oxidizing enzyme is a laccase enzyme.

14. The laccase-peptide complex according to claim 13, wherein the laccase is obtainable from a Stachybotrys species.

15. A laccase-peptide complex comprising a laccase obtainable from a Stachybotrys species and a peptide having an amino acid sequence illustrated in any one of SEQ ID NOS: 2 through 433.

16. The laccase-peptide complex of claim 15, wherein the peptide has an amino acid sequence illustrated in any one of SEQ ID NOs: 4, 16, 24, 92, 94, 104, 105, 120, 198, 233, 247, 279, 293, 300, 304, and 317.

17. The laccase-peptide complex of claim 15, wherein the laccase has the amino acid sequence illustrated in SEQ ID NO: 1 or a variant thereof, said variant having at least 75% sequence identity to the amino acid sequence illustrated in SEQ ID NO: 1 and said variant is capable of modifying the color associated with colored compounds.

18. The laccase-peptide complex of claim 17 comprising a variant of sequence SEQ ID NO: 1, wherein said variant differs from SEQ ID NO: 1 in at least one of the positions 48, 67, 70, 76, 83, 98, 115, 119, 134, 171, 175, 177, 179, 188, 236, 246, 253, 269, 272, 296, 302, 308, 318, 329, 331, 346, 348, 349, 365, 390, 391, 394, 404, 415, 423, 425, 428, 434, 465, 479, 481, 483, 499, 550, 562, 570, and 573 or sequence positions corresponding thereto and wherein said variant is capable of modifying the color associated with colored compounds.

19. The laccase-peptide complex of claim 18, wherein the laccase variant comprises a sequence that differs from that of SEQ ID NO: 1 in at least one of the positions 188, 254, 272, 346, 348, 394 and 425.

20. The laccase-peptide complex of claim 17, wherein the laccase has the amino acid sequence illustrated in SEQ ID NO: 1.

21. The laccase-peptide complex of claim 18, wherein the peptide is selected from the group consisting of peptides having the amino acid sequence illustrated in any one of SEQ ID NOs: 4, 16, 24, 92, 94, 104, 105, 120, 198, 233, 247, 279, 293, 300, 304, and 317.

22. An expression vector comprising a polynucleotide encoding the phenol oxidizing enzyme-peptide complex of claim 8.

23. A host cell comprising the vector of claim 22.

24. The host cell of claim 23, wherein said host cell is a fungal cell.

25. A laccase-peptide complex comprising a peptide having the amino acid sequence illustrated in any one of SEQ ID NOs:2-433 and a laccase, wherein said laccase comprises the amino acid sequence illustrated in SEQ ID NO: 1 or a variant thereof, wherein said variant differs in at least one of the positions 188, 254, 272, 346, 348, 394 and 425 of SEQ ID NO: 1.

26. A method of enhancing the binding of a laccase enzyme to a target stain comprising;
a) obtaining a peptide according to claim 1,
b) combining said peptide with a laccase to form a laccase-peptide complex, and
c) exposing the target stain to the laccase-peptide complex under suitable conditions to allow the complex to bind with the target stain.

27. The method according to claim 26, wherein the peptide is selected from the group consisting of SEQ ID NOS: 4, 16, 24, 92, 94, 104, 105, 120, 198, 233, 247, 279, 293, 300, 304, and 317.

28. The method according to claim 26, wherein the laccase is an enzymatically active laccase having the amino acid sequence illustrated in SEQ ID NO: 1 or a variant thereof, said variant having at least 75% sequence identity to the amino acid sequence illustrated in SEQ ID NO: 1 and which differs in at least one of the positions 48, 67, 70, 76, 83, 98, 115, 119, 134, 171, 175, 177, 179, 188, 236, 246, 253, 269, 272, 296, 302, 308, 318, 329, 331, 346, 348, 349, 365, 390, 391, 394, 404, 415, 423, 425, 428, 434, 465, 479, 481, 483, 499, 550, 562, 570, and 573 or sequence positions corresponding thereto and wherein said variant is capable of modifying the color associated with a targeted stain.

29. A detergent composition comprising
a) one or more surfactants and
b) the phenol oxidizing enzyme-peptide complex of claim 8, wherein said complex selectively binds to a target stain during a wash cycle that includes agitation.

30. The detergent composition of claim 29, wherein the phenol oxidizing enzyme is a laccase.

31. The detergent composition of claim 30 further comprising one or more enzymes other than laccase.

32. A method for removing stains from a fabric comprising contacting at least a part of a stained fabric with the detergent composition of claim 29.

33. An enzymatic composition comprising
a) one or more surfactants and
b) the phenol oxidizing enzyme-peptide complex of claim 8.

34. A method for producing a host cell comprising a polynucleotide encoding a laccase-peptide complex, comprising the steps of:
(a) obtaining a polynucleotide encoding a laccase having at least 68% identity to the amino acid sequence disclosed in SEQ ID NO: 1;
(b) obtaining a polynucleotide encoding a binding peptide having an amino acid sequence as illustrated in any one SEQ ID NOS: 2-433;
(c) conjugating the polynucleotide of step (a) with (b);
(d) introducing said conjugated polynucleotide into the host cell; and
(e) growing said host cell under conditions suitable for the production of said laccase-peptide complex.

35. The method of claim 34, wherein said conjugated polynucleotide is introduced on a replicating plasmid.

36. The method of claim 34, wherein said conjugated polynucleotide is integrated into the host cell genome.

37. A method of using a binding peptide to target a stain on a textile comprising
a) obtaining a binding peptide as illustrated in any one of SEQ ID NOS: 2-433;
b) exposing said binding peptide to a target stain, wherein said binding peptide binds to said stain and not to said textile.

38. The method according to claim 37, wherein the binding peptide is selected from the group consisting of SEQ ID NOS: 4, 16, 24, 92, 94, 104, 105, 120, 198, 233, 247, 279, 293, 300, 304, and 317.

39. A method of enhancing the selectivity of a phenol oxidizing enzyme to a target stain which comprises,
a) derivatizing a laccase with a binding peptide as illustrated in any one of SEQ ID NOS: 2-433 to form a laccase-peptide complex; and
b) exposing the laccase-peptide complex to a target stain, wherein selectivity of the laccase-peptide complex to the target stain is greater than the selectivity of a nonderivatized laccase having the same amino acid sequence as the laccase of the laccase-peptide complex.
